(19) United States
(12) Patent Application Publication
(10) Pub. No.: US 2019/0256502 A1
(43) Pub. Date: Aug. 22, 2019

(54) ORGANIC COMPOUNDS

(71) Applicant: HOX THERAPEUTICS LIMITED, Ashtead, Surrey (GB)

(72) Inventor: Richard MORGAN, Bradford, Yorkshire (GB)

(73) Assignee: HOX THERAPEUTICS LIMITED, Ashtead, Surrey (GB)

(21) Appl. No.: 16/333,536

(22) PCT Filed: Sep. 15, 2017
(86) PCT No.: PCT/GB2017/052739

§ 371 Date: Mar. 14, 2019

(30) Foreign Application Priority Data
Sep. 16, 2016 (GB) 1615797.6

(51) Int. Cl. C07D 409/06; A61K 31/4045; A61K 45/06

(52) U.S. Cl. CPC C07D 409/06; A61K 45/06; A61K 31/4045

(57) ABSTRACT

The present invention relates to compounds of Formula I or a pharmaceutically salt or solvate thereof, which impair PBX-dependent transcription regulation, particularly which affect the binding of HOX to PBX and their use in a number of applications, including the reduction of aberrant cell division, e.g. to treat certain cancers, and to maintain pluripotency of stem cells, e.g. to maintain the pluripotency of stem cells during culture expansion. wherein R¹, R² and R³ are as defined herein.

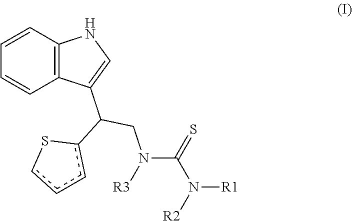

The present invention relates to novel indole derivatives which impair PBX-dependent transcription regulation, particularly which affect the binding of HOX to PBX and their use in a number of applications, including the reduction of aberrant cell division, e.g. to treat certain cancers, and to maintain pluripotency of stem cells, e.g. to maintain the pluripotency of stem cells during culture expansion.

A variety of transcription factors are involved in the regulation of expression of proteins during embryogenesis and adult stem cell maturation. Homeobox (HOX) genes contain a highly conserved nucleotide sequence of about 180 bp which encodes a homeodomain of about 60 amino acids. A homeodomain is a DNA-binding protein domain which can bind to target sequences in other genes and regulate their expression during development. The clustered Hox genes are key developmental regulators and are highly conserved throughout evolution. The homeotic HOX proteins which they encode function as transcription factors to control axial patterning by regulating the transcription of subordinate downstream genes, e.g. developmental genes. Pre-B-cell transformation related gene (PBX) is also an important regulatory protein that controls gene expression during development by interacting cooperatively with HOX to bind to the target DNA (Mann et al., 1996, Trends Genet., 12(7), p258-262).

It is known that by inhibiting the binding of PBX to its binding partners, aberrant cell growth may be reduced to prevent or treat disorders or conditions in which such cell growth occurs. Such inhibition has been found to have profound and useful effects on stem cells, which allows the pluripotency of these cells to be maintained. These findings offer significant clinical applications in which desired cells may be protected and possibly expanded whilst the growth of detrimental cells may be prevented (Morgan, R., Pirard, P. M., Shears, L., Sohal, J., Pettengell, R. & Pandha, H. S. (2007) Antagonism of HOX/PBX dimer formation blocks the in vivo proliferation of melanoma. Cancer Res, 67, 5806-5813; Shears, L., Plowright, L., Harrington, K., Pandha, H. S. & Morgan, R. (2008) Disrupting the interaction between HOX and PBX causes necrotic and apoptotic cell death in the renal cancer lines CaKi-2 and 769-P. J Urol, 180, 2196-2201; Plowright, L., Harrington, K. J., Pandha, H. S. & Morgan, R. (2009) HOX transcription factors are potential therapeutic targets in non-small-cell lung cancer (targeting HOX genes in lung cancer). Br J Cancer, 100, 470-475; Daniels, T. R., Neacato, II, Rodriguez, J. A., Pandha, H. S., Morgan, R. & Penichet, M. L. (2010) Disruption of HOX activity leads to cell death that can be enhanced by the interference of iron uptake in malignant B cells. Leukemia, 24, 1555-1565; Morgan, R., Plowright, L., Harrington, K. J., Michael, A. & Pandha, H. S. (2010) Targeting HOX and PBX transcription factors in ovarian cancer. BMC Cancer, 10, 89; Morgan, R., Boxall, A., Harrington, K. J., Simpson, G. R., Gillett, C., Michael, A. & Pandha, H. S. (2012) Targeting the HOX/PBX dimer in breast cancer. Breast Cancer Res Treat, 136, 389-398; Errico, M. C., Felicetti, F., Bottero, L., Mattia, G., Boe, A., Felli, N., Petrini, M., Bellenghi, M., Pandha, H. S., Calvaruso, M., Tripodo, C., Colombo, M. P., Morgan, R. & Care, A. (2013) The abrogation of the HOXB7/PBX2 complex induces apoptosis in melanoma through the miR-221&222-c-FOS pathway. Int J Cancer, 133, 879-892; Morgan, R., Boxall, A., Harrington, K. J., Simpson, G. R., Michael, A. & Pandha, H. S. (2014) Targeting HOX transcription factors in prostate cancer. BMC Urol, 14, 17; the contents of which are incorporated in their entirety for all purposes).

The present invention provides compounds which impair PBX-dependent transcription regulation (e.g. activation or repression), e.g. by interfering with the interaction between PBX and its co-factors, preferably HOX, and its target DNA, e.g. which affect the binding of HOX and PBX proteins, have downstream effects which can offer great advantages such as preventing or reducing aberrant cell division and maintaining pluripotency of stem cells. In particular, the present invention provides novel compounds which act as PBX modulators, in particular antagonists, more particularly, of the binding of the hexapeptide region of HOX proteins to PBX.

In one aspect, the invention provides a compound of Formula I:

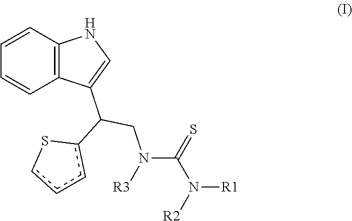

or a pharmaceutically salt or solvate thereof, wherein
R1 is selected from H, C₁-C₈ alkyl; C₃-C₁₀ cycloalkyl; C₂-C₈ alkenyl; C₅-C₁₀-cycloalkenyl; C₂-C₈ alkynyl; C₆-C₁₅ aryl; (C₁-C₄ alkyl)-(C₃-C₁₀ cycloalkyl); (C₁-C₄ alkyl)-(C₂-C₈ alkenyl); (C₁-C₄ alkyl)-(C₅-C₁₀ cycloalkenyl); (C₁-C₄ alkyl)-(C₂-C₈ alkynyl); (C₁-C₄ alkyl)-(C₆-C₁₅ aryl);
R² and R³ are independently selected from H and C₁-C₃alkyl;
and [symbol] denotes a single or double bond, with the proviso that a maximum of two double bonds are present, which cannot be adjacent to each other.

In one embodiment of the invention as defined anywhere above, R¹ is selected from C₁-C₈ alkyl; C₃-C₁₀ cycloalkyl; C₂-C₈ alkenyl; C₅-C₁₀-cycloalkenyl; C₂-C₈ alkynyl; (C₁-C₄ alkyl)-(C₃-C₁₀ cycloalkyl); (C₁-C₄ alkyl)-(C₂-C₈ alkenyl); (C₁-C₄ alkyl)-(C₅-C₁₀ cycloalkenyl); (C₁-C₄ alkyl)-(C₂-C₈ alkynyl).

In a further embodiment of the invention as defined anywhere above, R¹ is selected from C₁-C₈ alkyl; (C₁-C₄ alkyl)-(C₃-C₈ cycloalkyl); (C₁-C₄ alkyl)-(C₂-C₈ alkenyl); (C₁-C₄ alkyl)-(C₅-C₁₀ cycloalkenyl); (C₁-C₄ alkyl)-(C₂-C₈ alkynyl).

In a yet further embodiment of the invention as defined anywhere above, R¹ is selected from C₁-C₈ alkyl; (C₁-C₄ alkyl)-(C₃-C₈ cycloalkyl); (C₁-C₄ alkyl)-(C₂-C₈ alkenyl).

In a yet further embodiment of the invention as defined anywhere above, R¹ is C₁-C₈ alkyl.

In a yet further embodiment of the invention as defined anywhere above, R¹ is C₅-C₇ alkyl.

In a yet further embodiment of the invention as defined anywhere above, R¹ is n-pentyl

In a yet further embodiment of the invention as defined anywhere above, R¹ is n-hexyl

In a yet further embodiment of the invention as defined anywhere above, R¹ is n-heptyl

In another further embodiment of the invention as defined anywhere above, R¹ is (C₁-C₄ alkyl)-(C₃-C₈ cycloalkyl)

In another embodiment of the invention as defined anywhere above, R² is H.

In another embodiment of the invention as defined anywhere above, R³ is H.

In a yet further embodiment, the invention provides a compound of Formula I′

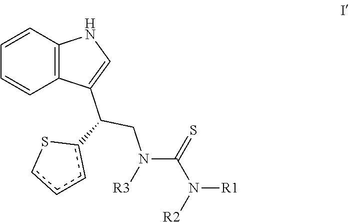

or a pharmaceutically acceptable salt or solvate thereof wherein R¹, R² and R³ are as defined anywhere hereinabove in respect of a compound of Formula I.

In a yet further embodiment, the invention provides a compound of Formula Ia

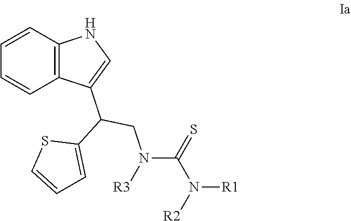

or a pharmaceutically acceptable salt or solvate thereof wherein R¹, R² and R³ are as defined anywhere hereinabove in respect of a compound of Formula I.

In a yet further embodiment, the invention provides a compound of Formula Ia′

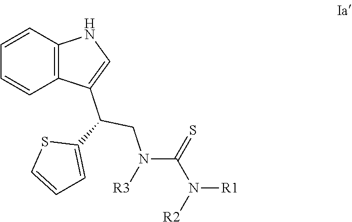

or a pharmaceutically acceptable salt or solvate thereof wherein R¹, R² and R³ are as defined anywhere hereinabove in respect of a compound of Formula I.

In a yet further embodiment, the invention provides a compound of Formula Ib

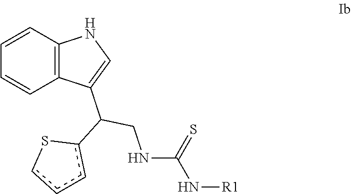

or a pharmaceutically acceptable salt or solvate thereof wherein R¹ is as defined anywhere hereinabove in respect of a compound of Formula I.

In a yet further embodiment, the invention provides a compound of Formula Ib′

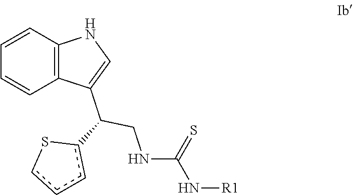

or a pharmaceutically acceptable salt or solvate thereof wherein R¹ is as defined anywhere hereinabove in respect of a compound of Formula I.

In a yet further embodiment, the invention provides a compound of Formula Ic

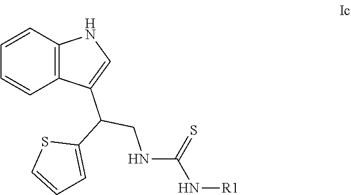

or a pharmaceutically acceptable salt or solvate thereof wherein R¹ is as defined anywhere hereinabove in respect of a compound of Formula I.

In a yet further embodiment, the invention provides a compound of Formula Ic′

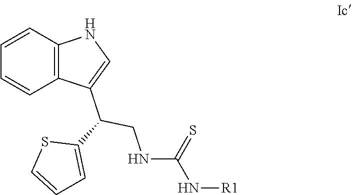

or a pharmaceutically acceptable salt or solvate thereof wherein R¹ is as defined anywhere hereinabove in respect of a compound of Formula I.

In another embodiment, individual compounds according to the invention are those listed in the Examples section below.

In another embodiment of the invention, there is provided a compound according to the invention which is selected from Examples 1, 2, 3 and 4 or a pharmaceutically acceptable salt or solvate thereof.

In another embodiment of the invention, there is provided a compound according to the invention which is selected from:
1-heptyl-3-[(2-(1H-indol-3-yl)-2-(2-thienyl)ethyl]thiourea;
1-hexyl-3-[(2-(1H-indol-3-yl)-2-(2-thienyl)ethyl]thiourea;
1-pentyl-3-[(2-(1H-indol-3-yl)-2-(2-thienyl)ethyl]thiourea;
1-butyl-3-[2-(1H-indol-3-yl)-2-(2-thienyl)ethyl]thiourea;
1-propyl-3-[2-(1H-indol-3-yl)-2-(2-thienyl)ethyl]thiourea;
1-ethyl-3-[(2-(1H-indol-3-yl)-2-(2-thienyl)ethyl]thiourea;
1-methyl-3-[2-(1H-indol-3-yl)-2-(2-thienyl)ethyl]thiourea;
1-cyclohexylmethyl-3-[2-(1H-indol-3-yl)-2-(2-thienyl)ethyl]thiourea; and
1-[2-(1H-indol-3-yl)-2-(2-thienyl)ethyl]-3-(2-methylallyl)thiourea;
   or a pharmaceutically acceptable salt or solvate thereof.

In a further embodiment of the invention, there is provided a compound according to the invention which is selected from:
1-heptyl-3-[(2-(1H-indol-3-yl)-2-(2-thienyl)ethyl]thiourea;
1-hexyl-3-[(2-(1H-indol-3-yl)-2-(2-thienyl)ethyl]thiourea; and
1-pentyl-3-[(2-(1H-indol-3-yl)-2-(2-thienyl)ethyl]thiourea;
   or a pharmaceutically acceptable salt or solvate thereof.

In another embodiment of the invention, there is provided a compound according to the invention which is selected from:
1-heptyl-3-[((2R-2-(1H-indol-3-yl)-2-(2-thienyl)ethyl]thiourea;
1-hexyl-3-[((2R)-2-(1H-indol-3-yl)-2-(2-thienyl)ethyl]thiourea;
1-pentyl-3-[((2R)-2-(1H-indol-3-yl)-2-(2-thienyl)ethyl]thiourea;
1-butyl-3-[(2R)-2-(1H-indol-3-yl)-2-(2-thienyl)ethyl]thiourea;
1-propyl-3-[(2R)-2-(1H-indol-3-yl)-2-(2-thienyl)ethyl]thiourea;
1-ethyl-3-[(2R)-2-(1H-indol-3-yl)-2-(2-thienyl)ethyl]thiourea;
1-cyclohexylmethyl-3-[(2R)-2-(1H-indol-3-yl)-2-(2-thienyl)ethyl]thiourea
1-methyl-3-[(2R)-2-(1H-indol-3-yl)-2-(2-thienyl)ethyl]thiourea; and
1-[(2R)-2-(1H-indol-3-yl)-2-(2-thienyl)ethyl]-3-(2-methylallyl)thiourea;
   or a pharmaceutically acceptable salt or solvate thereof.

In a further embodiment of the invention, there is provided a compound according to the invention which is selected from:
1-heptyl-3-[((2R-2-(1H-indol-3-yl)-2-(2-thienyl)ethyl]thiourea;
1-hexyl-3-[((2R)-2-(1H-indol-3-yl)-2-(2-thienyl)ethyl]thiourea; and
1-pentyl-3-[((2R)-2-(1H-indol-3-yl)-2-(2-thienyl)ethyl]thiourea;
   or a pharmaceutically acceptable salt or solvate thereof.

In the embodiments mentioned herein, where only certain variables are defined, it is intended that the remainder of the variables are as defined in any embodiment herein. Thus, the invention provides for the combination of limited or optional definitions of variables.

A skilled person will appreciate that

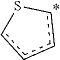

denotes thiophenyl; 2,3-dihydrothiophenyl; 2,5-dihydrothiophenyl; 4,5-dihydrothiophenyl; and tetrahydrothiopheny; wherein * denotes the point of attachment.

The following terms as used herein are intended to have the following meanings:

“Optionally substituted” as used herein means the group referred to can be unsubstituted, or substituted at one or two or three positions by any one or any combination of the radicals listed thereafter.

“C₁-C₃ alkyl”, “C₁-C₆ alkyl”, “C₁-C₈ alkyl” and the like, as used herein, denotes a straight chain or branched alkyl group that contains one to three, six or eight (or the relevant number) carbon atoms and which may be substituted as defined.

“C₂-C₆ alkenyl”, “C₂-C₈ alkenyl” and the like, as used herein, denotes a straight chain or branched alkenyl group that contains two to six or eight (or the relevant number) carbon atoms and which may be substituted as defined.

“C₂-C₆ alkynyl”, “C₂-C₈ alkynyl” and the like, as used herein, denotes a straight chain or branched alkynyl group that contains two to six or eight (or the relevant number) carbon atoms and which may be substituted as defined.

“Aryl”, as used herein, represents an aromatic carbocyclic ring system having 6 to 15 carbon atoms. It can be monocyclic, bicyclic or tricyclic, and may be optionally substituted as defined. Examples of C₆-C₁₅-aryl groups include but are not limited to phenyl, phenylene, benzenetriyl, indanyl, naphthyl, naphthylene, naphthalenetriyl and anthracenyl.

“C₃-C₁₀-cycloalkyl” denotes a fully saturated carbocyclic ring having 3 to 10 ring carbon atoms, for example a monocyclic group such as a cyclopropyl, cyclobutyl, cyclopentyl or cyclohexyl, cycloheptyl, cyclooctyl, cyclononyl or cyclodecyl, or a bicyclic group such as bicycloheptyl or bicyclooctyl. Different numbers of carbon atoms may be specified, with the definition being amended accordingly. The cycloalkyl group can be substituted or unsubstituted.

“C₅-C₁₀-cycloalkenyl” denotes a partially saturated carbocyclic ring having 5 to 10 ring carbon atoms, for example a monocyclic group such as a cyclopentenyl or cyclohexenyl, cycloheptenyl, cyclooctenyl or cyclononenyl, or a bicyclic group such as bicycloheptenyl or bicyclooctenyl. The ring or ring system may contain more than one carbon-carbon double bond. Different numbers of carbon atoms may be specified, with the definition being amended accordingly. The cycloalkenyl group can be substituted or unsubstituted.

Throughout this specification and in the claims that follow, unless the context requires otherwise, the word “comprise”, or variations such as “comprises” or “comprising”, should be understood to imply the inclusion of a stated integer or step or group of integers or steps but not the exclusion of any other integer or step or group of integers or steps.

As used herein, the term “pharmaceutically acceptable salts” refers to salts that retain the biological effectiveness and properties of the compounds of this invention and, which typically are not biologically or otherwise undesirable. In many cases, the compounds of the present invention are capable of forming acid and/or base salts by virtue of the presence of amino and/or carboxyl groups or groups similar thereto.

Pharmaceutically acceptable acid addition salts can be formed with inorganic acids and organic acids, e.g., acetate, aspartate, benzoate, besylate, bromide/hydrobromide, bicarbonate/carbonate, bisulfate/sulfate, cam phorsulfornate, chloride/hydrochloride, chlortheophyllonate, citrate, ethandisulfonate, fumarate, gluceptate, gluconate, glucuronate, hippurate, hydroiodide/iodide, isethionate, lactate, lactobionate, laurylsulfate, malate, maleate, malonate, mandelate, mesylate, methylsulphate, naphthoate, napsylate, nicotinate, nitrate, octadecanoate, oleate, oxalate, palmitate, pamoate, phosphate/hydrogen phosphate/dihydrogen phosphate, polygalacturonate, propionate, stearate, succinate, sulfosalicylate, tartrate, tosylate, trifluoroacetate and trifluoromethylsulfonate salts.

Inorganic acids from which salts can be derived include, for example, hydrochloric acid, hydrobromic acid, sulfuric acid, nitric acid, phosphoric acid, and the like. Organic acids from which salts can be derived include, for example, acetic acid, propionic acid, glycolic acid, oxalic acid, maleic acid, malonic acid, succinic acid, fumaric acid, tartaric acid, citric acid, benzoic acid, mandelic acid, methanesulfonic acid, ethanesulfonic acid, toluenesulfonic acid, trifluoromethylsulfonic acid, sulfosalicylic acid, and the like. Pharmaceutically acceptable base addition salts can be formed with inorganic and organic bases.

Inorganic bases from which salts can be derived include, for example, ammonium salts and metals from columns I to XII of the periodic table. In certain embodiments, the salts are derived from sodium, potassium, ammonium, calcium, magnesium, iron, silver, zinc, and copper; particularly suitable salts include ammonium, potassium, sodium, calcium and magnesium salts.

Organic bases from which salts can be derived include, for example, primary, secondary, and tertiary amines, substituted amines including naturally occurring substituted amines, cyclic amines, basic ion exchange resins, and the like. Certain organic amines include isopropylamine, benzathine, cholinate, diethanolamine, diethylamine, lysine, meglumine, piperazine and tromethamine.

Furthermore, the compounds of the present invention, including their salts, can also be obtained in the form of their hydrates, or include other solvents used for their crystallization.

Compounds of the invention that contain groups capable of acting as donors and/or acceptors for hydrogen bonds may be capable of forming co-crystals with suitable co-crystal formers. These co-crystals may be prepared from compounds of formula (I) by known co-crystal forming procedures. Such procedures include grinding, heating, co-subliming, co-melting, or contacting in solution compounds of formula (I) with the co-crystal former under crystallization conditions and isolating co-crystals thereby formed. Suitable co-crystal formers include those described in WO 2004/078163. Hence the invention further provides co-crystals comprising a compound of formula (I).

As used herein, the term “isomers” refers to different compounds that have the same molecular formula but differ in arrangement and configuration of the atoms. Also as used herein, the term “an optical isomer” or “a stereoisomer” refers to any of the various stereo isomeric configurations which may exist for a given compound of the present invention and includes geometric isomers. It is understood that a substituent may be attached at a chiral center of a carbon atom. Therefore, the invention includes enantiomers, diastereomers or racemates of the compound. “Enantiomers” are a pair of stereoisomers that are non-superimposable mirror images of each other. A 1:1 mixture of a pair of enantiomers is a “racemic” mixture. The term is used to designate a racemic mixture where appropriate. “Diastereoisomers” are stereoisomers that have at least two asymmetric atoms, but which are not mirror-images of each other. The absolute stereochemistry is specified according to the Cahn-Ingold- Prelog R-S system. When a compound is a pure enantiomer the stereochemistry at each chiral carbon may be specified by either R or S. Resolved compounds whose absolute configuration is unknown can be designated (+) or (−) depending on the direction (dextro- or levorotatory) which they rotate plane polarized light at the wavelength of the sodium D line. Certain of the compounds described herein contain one or more asymmetric centers or axes and may thus give rise to enantiomers, diastereomers, and other stereoisomeric forms that may be defined, in terms of absolute stereochemistry, as (R)- or (S)-. The present invention is meant to include all such possible isomers, including racemic mixtures, optically pure forms and intermediate mixtures. Optically active (R)- and (S)-isomers may be prepared using chiral synthons or chiral reagents, or resolved using conventional techniques. If the compound contains a double bond, the substituent may be E or Z configuration. If the compound contains a disubstituted cycloalkyl, the cycloalkyl substituent may have a cis- or trans-configuration. All tautomeric forms are also intended to be included. Tautomers are one of two or more structural isomers that exist in equilibrium and are readily converted from one isomeric form to another. Examples of tautomers include but are not limited to those compounds defined in the claims.

Any asymmetric atom (e.g., carbon or the like) of the compound(s) of the present invention can be present in racemic or enantiomerically enriched, for example the (R)-, (S)- or (R,S)-configuration. In certain embodiments, each asymmetric atom has at least 50% enantiomeric excess, at least 60% enantiomeric excess, at least 70% enantiomeric excess, at least 80% enantiomeric excess, at least 90% enantiomeric excess, at least 95% enantiomeric excess, or at least 99% enantiomeric excess in the (R)- or (S)-configuration. Substituents at atoms with unsaturated bonds may, if possible, be present in cis- (Z)- or trans-(E)-form.

Accordingly, as used herein a compound of the present invention can be in the form of one of the possible isomers, rotamers, atropisomers, tautomers or mixtures thereof, for example, as substantially pure geometric (cis or trans) isomers, diastereomers, optical isomers (antipodes), racemates or mixtures thereof.

Any resulting mixtures of isomers can be separated on the basis of the physicochemical differences of the constituents, into the pure or substantially pure geometric or optical isomers, diastereomers, racemates, for example, by chromatography and/or fractional crystallization.

Any resulting racemates of final products or intermediates can be resolved into the optical antipodes by known methods, e.g., by separation of the diastereomeric salts thereof, obtained with an optically active acid or base, and liberating the optically active acidic or basic compound. In particular, a basic moiety may thus be employed to resolve the compounds of the present invention into their optical antipodes, e.g., by fractional crystallization of a salt formed with an optically active acid, e.g., tartaric acid, dibenzoyl tartaric acid, diacetyl tartaric acid, di-O,O′-p-toluoyl tartaric acid, mandelic acid, malic acid or camphor-10-sulfonic acid. Racemic products can also be resolved by chiral chromatography, e.g., high pressure liquid chromatography (HPLC) using a chiral adsorbent.

Since the compounds of the invention are intended for use in pharmaceutical compositions it will readily be understood that they are each preferably provided in substantially pure form, for example at least 60% pure, more suitably at least 75% pure and preferably at least 85%, especially at least 98% pure (% are on a weight for weight basis). Impure preparations of the compounds may be used for preparing the more pure forms used in the pharmaceutical compositions; these less pure preparations of the compounds should contain at least 1%, more suitably at least 5% and preferably from 10 to 59% of a compound of the invention.

When both a basic group and an acid group are present in the same molecule, the compounds of the present invention may also form internal salts, e.g., zwitterionic molecules.

A pro-drug is an active or inactive compound that is modified chemically through in vivo physiological action, such as hydrolysis, metabolism and the like, into a compound of the invention following administration of the prodrug to a subject. The compounds of the present invention may themselves be active and/or act as prodrugs which convert in vivo to active compounds. The suitability and techniques involved in making and using pro-drugs are well known by those skilled in the art. Prodrugs can be conceptually divided into two non-exclusive categories, bioprecursor prodrugs and carrier prodrugs. See The Practice of Medicinal Chemistry, Ch. 31-32 (Ed. Wermuth, Academic Press, San Diego, Calif., 2001). Generally, bioprecursor prodrugs are compounds, which are inactive or have low activity compared to the corresponding active drug compound, that contain one or more protective groups and are converted to an active form by metabolism or solvolysis. Both the active drug form and any released metabolic products should have acceptably low toxicity. Carrier prodrugs are drug compounds that contain a transport moiety, e.g., that improve uptake and/or localized delivery to a site(s) of action. Desirably for such a carrier prodrug, the linkage between the drug moiety and the transport moiety is a covalent bond, the prodrug is inactive or less active than the drug compound, and any released transport moiety is acceptably non-toxic. For prodrugs where the transport moiety is intended to enhance uptake, typically the release of the transport moiety should be rapid. In other cases, it is desirable to utilize a moiety that provides slow release, e.g., certain polymers or other moieties, such as cyclodextrins. Carrier prodrugs can, for example, be used to improve one or more of the following properties: increased lipophilicity, increased duration of pharmacological effects, increased site-specificity, decreased toxicity and adverse reactions, and/or improvement in drug formulation (e.g., stability, water solubility, suppression of an undesirable organoleptic or physiochemical property). For example, lipophilicity can be increased by esterification of (a) hydroxyl groups with lipophilic carboxylic acids (e.g., a carboxylic acid having at least one lipophilic moiety), or (b) carboxylic acid groups with lipophilic alcohols (e.g., an alcohol having at least one lipophilic moiety, for example aliphatic alcohols). Exemplary prodrugs are, e.g., esters of free carboxylic acids and S-acyl derivatives of thiols and O-acyl derivatives of alcohols or phenols, wherein acyl has a meaning as defined herein. Suitable prodrugs are often pharmaceutically acceptable ester derivatives convertible by solvolysis under physiological conditions to the parent carboxylic acid, e.g., lower alkyl esters, cycloalkyl esters, lower alkenyl esters, benzyl esters, mono- or di-substituted lower alkyl esters, such as the ω-(amino, mono- or di-lower alkylamino, carboxy, lower alkoxycarbonyl)-lower alkyl esters, the α-(lower alkanoyloxy, lower alkoxycarbonyl or di-lower alkylaminocarbonyl)-lower alkyl esters, such as the pivaloyloxymethyl ester and the like conventionally used in the art. In addition, amines have been masked as arylcarbonyloxymethyl substituted derivatives which are cleaved by esterases in vivo releasing the free drug and formaldehyde (Bundgaard, J. Med. Chem. 2503 (1989)). Moreover, drugs containing an acidic NH group, such as imidazole, imide, indole and the like, have been masked with N-acyloxymethyl groups (Bundgaard, Design of Prodrugs, Elsevier (1985)). Hydroxy groups have been masked as esters and ethers. EP 039,051 (Sloan and Little) discloses Mannich-base hydroxamic acid prodrugs, their preparation and use.

Any formula given herein is also intended to represent unlabeled forms as well as isotopically labeled forms of the compounds. Isotopically labeled compounds have structures depicted by the formulas given herein except that one or more atoms are replaced by an atom having a selected atomic mass or mass number. Examples of isotopes that can be incorporated into compounds of the invention include isotopes of hydrogen, carbon, nitrogen, oxygen, phosphorous, fluorine, and chlorine, such as ²H, ³H, ¹¹C, ¹³C, ¹⁴C, ¹⁵N, ¹⁸F, ³¹P, ³⁵S, ³⁸Cl, ¹²⁵I respectively. The invention includes various isotopically labeled compounds as defined herein, for example those into which radioactive isotopes, such as ³H, and ¹⁴C, are present. Such isotopically labelled compounds are useful in metabolic studies (with ¹⁴C), reaction kinetic studies (with, for example ²H or ³H), detection or imaging techniques, such as positron emission tomography (PET) or single-photon emission computed tomography (SPECT) including drug or substrate tissue distribution assays, or in radioactive treatment of patients. In particular, an ¹⁸F or labeled compound may be particularly desirable for PET or SPECT studies. Isotopically labeled compounds of this invention and prodrugs thereof can generally be prepared by carrying out the procedures disclosed in the schemes or in the examples and preparations described below by substituting a readily available isotopically labeled reagent for a non-isotopically labeled reagent.

Further, substitution with heavier isotopes, particularly deuterium (i.e., ²H or D) may afford certain therapeutic advantages resulting from greater metabolic stability, for example increased in vivo half-life or reduced dosage requirements or an improvement in therapeutic index. It is understood that deuterium in this context is regarded as a substituent of a compound of the formula (I). The concentration of such a heavier isotope, specifically deuterium, may be defined by the isotopic enrichment factor. The term “isotopic enrichment factor” as used herein means the ratio between the isotopic abundance and the natural abundance of a specified isotope. If a substituent in a compound of this invention is denoted deuterium, such compound has an isotopic enrichment factor for each designated deuterium atom of at least 3500 (52.5% deuterium incorporation at each designated deuterium atom), at least 4000 (60% deuterium incorporation), at least 4500 (67.5% deuterium incorporation), at least 5000 (75% deuterium incorporation), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation).

Isotopically-labeled compounds of formula (I) can generally be prepared by conventional techniques known to those skilled in the art or by processes analogous to those described in the accompanying Examples and Preparations using an appropriate isotopically-labeled reagents in place of the non-labeled reagent previously employed.

Pharmaceutically acceptable solvates in accordance with the invention include those wherein the solvent of crystallization may be isotopically substituted, e.g. D₂O, d₆-acetone, d₆-DMSO.

### Synthesis

The compounds of the invention may be synthesized by the general synthetic routes below, specific examples of which are described in more detail in the Examples.

Compounds of formula I may be prepared according to Scheme 1.

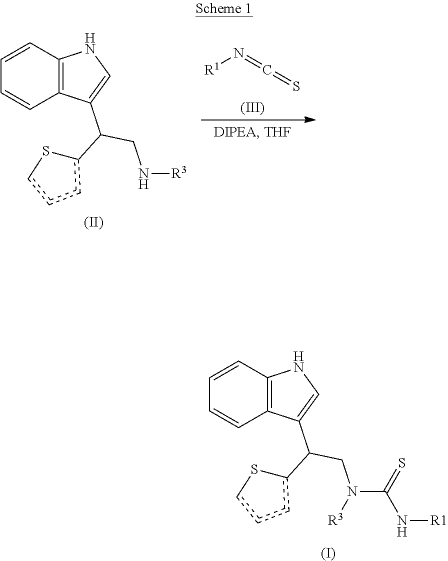

Alternatively, compounds of formula I may be prepared according to Scheme 2.

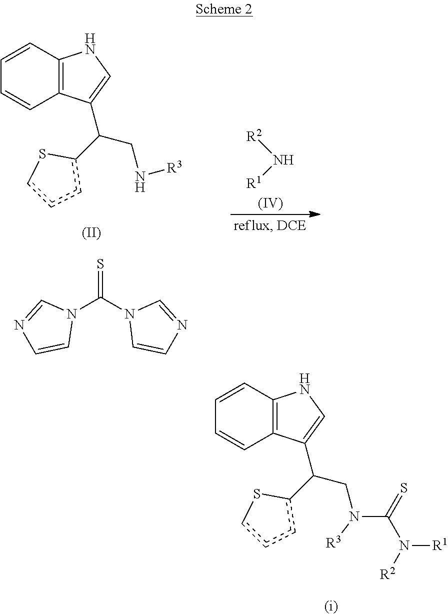

The above general schemes may be used to prepare compounds of the present invention. The desired specific compounds can be prepared by selecting the appropriate starting materials, reactants and reaction conditions.

The starting materials and reagents in the above scheme are all either available commercially or can be prepared following literature precedents.

Within the scope of this text, only a readily removable group that is not a constituent of the particular desired end product of the compounds of the present invention is designated a “protecting group”, unless the context indicates otherwise. The protection of functional groups by such protecting groups, the protecting groups themselves, and their cleavage reactions are described for example in standard reference works, such as J. F. W. McOmie, “Protective Groups in Organic Chemistry”, Plenum Press, London and New York 1973, in T. W. Greene and P. G. M. Wuts, “Protective Groups in Organic Synthesis”, Third edition, Wiley, New York 1999, in “The Peptides”; Volume 3 (editors: E. Gross and J. Meienhofer), Academic Press, London and New York 1981, in “Methoden der organischen Chemie” (Methods of Organic Chemistry), Houben Weyl, 4th edition, Volume 15/I, Georg Thieme Verlag, Stuttgart 1974, in H.-D. Jakubke and H. Jeschkeit, “Aminosäuren, Peptide, Proteine” (Amino acids, Peptides, Proteins), Verlag Chemie, Weinheim, Deerfield Beach, and Basel 1982, and in Jochen Lehmann, “Chemie der Kohlenhydrate: Monosaccharide and Derivate” (Chemistry of Carbohydrates: Monosaccharides and Derivatives), Georg Thieme Verlag, Stuttgart 1974. A characteristic of protecting groups is that they can be removed readily (i.e. without the occurrence of undesired secondary reactions) for example by solvolysis, reduction, photolysis or alternatively under physiological conditions (e.g. by enzymatic cleavage). Salts of compounds of the present invention having at least one salt-forming group may be prepared in a manner known to those skilled in the art. For example, salts of compounds of the present invention having acid groups may be formed, for example, by treating the compounds with metal compounds, such as alkali metal salts of suitable organic carboxylic acids, e.g. the sodium salt of 2-ethylhexanoic acid, with organic alkali metal or alkaline earth metal compounds, such as the corresponding hydroxides, carbonates or hydrogen carbonates, such as sodium or potassium hydroxide, carbonate or hydrogen carbonate, with corresponding calcium compounds or with ammonia or a suitable organic amine, stoichiometric amounts or only a small excess of the salt-forming agent preferably being used. Acid addition salts of compounds of the present invention are obtained in customary manner, e.g. by treating the compounds with an acid or a suitable anion exchange reagent. Internal salts of compounds of the present invention containing acid and basic salt-forming groups, e.g. a free carboxy group and a free amino group, may be formed, e.g. by the neutralisation of salts, such as acid addition salts, to the isoelectric point, e.g. with weak bases, or by treatment with ion exchangers. Salts can be converted into the free compounds in accordance with methods known to those skilled in the art. Metal and ammonium salts can be converted, for example, by treatment with suitable acids, and acid addition salts, for example, by treatment with a suitable basic agent.

Mixtures of isomers obtainable according to the invention can be separated in a manner known to those skilled in the art into the individual isomers; diastereoisomers can be separated, for example, by partitioning between polyphasic solvent mixtures, recrystallisation and/or chromatographic separation, for example over silica gel or by e.g. medium pressure liquid chromatography over a reversed phase column, and racemates can be separated, for example, by the formation of salts with optically pure salt-forming reagents and separation of the mixture of diastereoisomers so obtainable, for example by means of fractional crystallisation, or by chromatography over optically active column materials.

Intermediates and final products can be worked up and/or purified according to standard methods, e.g. using chromatographic methods, distribution methods, (re-) crystallization, and the like.

The following applies in general to all processes mentioned herein before and hereinafter.

All the above-mentioned process steps can be carried out under reaction conditions that are known to those skilled in the art, including those mentioned specifically, in the absence or, customarily, in the presence of solvents or diluents, including, for example, solvents or diluents that are inert towards the reagents used and dissolve them, in the absence or presence of catalysts, condensation or neutralizing agents, for example ion exchangers, such as cation exchangers, e.g. in the H+ form, depending on the nature of the reaction and/or of the reactants at reduced, normal or elevated temperature, for example in a temperature range of from about −100° C. to about 190° C., including, for example, from approximately −80° C. to approximately 150° C., for example at from −80 to −60° C., at room temperature, at from −20 to 40° C. or at reflux temperature, under atmospheric pressure or in a closed vessel, where appropriate under pressure, and/or in an inert atmosphere, for example under an argon or nitrogen atmosphere.

At all stages of the reactions, mixtures of isomers that are formed can be separated into the individual isomers, for example diastereoisomers or enantiomers, or into any desired mixtures of isomers, for example racemates or mixtures of diastereoisomers, for example analogously to the methods described under “Additional process steps”.

The solvents from which those solvents that are suitable for any particular reaction may be selected include those mentioned specifically or, for example, water, esters, such as lower alkyl-lower alkanoates, for example ethyl acetate, ethers, such as aliphatic ethers, for example diethyl ether, or cyclic ethers, for example tetrahydrofuran or dioxane, liquid aromatic hydrocarbons, such as benzene or toluene, alcohols, such as methanol, ethanol or 1- or 2-propanol, nitriles, such as acetonitrile, halogenated hydrocarbons, such as methylene chloride or chloroform, acid amides, such as dimethylformamide or dimethyl acetamide, bases, such as heterocyclic nitrogen bases, for example pyridine or N-methylpyrrolidin-2-one, carboxylic acid anhydrides, such as lower alkanoic acid anhydrides, for example acetic anhydride, cyclic, linear or branched hydrocarbons, such as cyclohexane, hexane or isopentane, methycyclohexane, or mixtures of those solvents, for example aqueous solutions, unless otherwise indicated in the description of the processes. Such solvent mixtures may also be used in working up, for example by chromatography or partitioning.

The compounds, including their salts, may also be obtained in the form of hydrates, or their crystals may, for example, include the solvent used for crystallization. Different crystalline forms may be present.

The invention relates also to those forms of the process in which a compound obtainable as an intermediate at any stage of the process is used as starting material and the remaining process steps are carried out, or in which a starting material is formed under the reaction conditions or is used in the form of a derivative, for example in a protected form or in the form of a salt, or a compound obtainable by the process according to the invention is produced under the process conditions and processed further in situ. All starting materials, building blocks, reagents, acids, bases, dehydrating agents, solvents and catalysts utilized to synthesize the compounds of the present invention are either commercially available or can be produced by organic synthesis methods known to one of ordinary skill in the art (Houben-Weyl 4^{th} Ed. 1952, Methods of Organic Synthesis, Thieme, Volume 21).

As a further aspect of the present invention, there is also provided a process for the preparation of compounds of formula I or a pharmaceutically acceptable salt or solvate thereof.

According to a further aspect of the invention there is provided a process of preparing a compound of the present invention or a pharmaceutically acceptable salt or solvate thereof comprising the step of:
a) reacting an amine compound of formula II

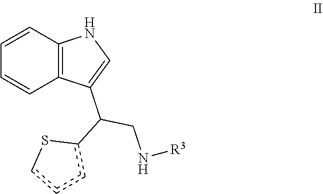

with an isothiocyanate compound of formula III (R¹—NCS) in the presence of DIPEA under conventional conditions wherein R¹ and R³ are as defined in respect of a compound of formula (I); or
b) reacting an amine compound of formula II

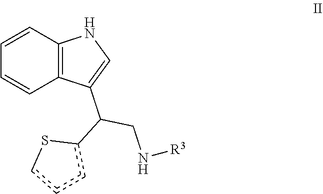

with thiocarbonyl diimidazole to yield an intermediate of formula V

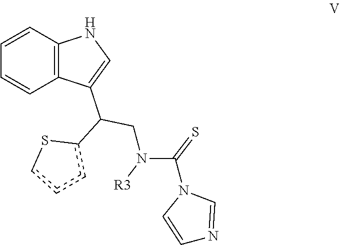

and reacting the intermediate of formula V with an amine compound of formula IV (R¹R²—NH) under conventional conditions wherein R¹, R² and R³ are as defined in respect of a compound of formula (I).

In one embodiment, the process further comprises converting the intermediate of formula (V) to an imidazolium salt prior to reacting with the amine compound of formula IV.

The invention further includes any variant of the present processes, in which an intermediate product obtainable at any stage thereof is used as starting material and the remaining steps are carried out, or in which the starting materials are formed in situ under the reaction conditions, or in which the reaction components are used in the form of their salts or optically pure antipodes.

Compounds of the invention and intermediates can also be converted into each other according to methods generally known to those skilled in the art.

The compounds of the invention may be used to block interactions of PBX with its binding partners, e.g. HOX, and preferably thereby prevent the binding of HOX to its target DNA. Thus in a further aspect the present invention provides use of a compound of formula I to reduce or inhibit binding of PBX to a binding partner, in particular HOX, or the use of such compounds to reduce or inhibit binding of HOX to its target DNA.

“PBX” refers to the protein products of the family of pre-B-cell transformation related genes and includes genes encoding extradenticle homeoprotein proteins and homologues of the Drosophila extradenticle gene, such as genes in vertebrates. Vertebrate PBX proteins are transcription factors that contain a homeodornain (Mann et al., 1996).

“HOX” refers to the protein products of the homeobox genes which contain a sequence which encodes a homeodomain of about 60 amino acids and a sequence which encodes the hexapeptide sequence N-terminal to the homeodomain (Morgan et al., 2000, TIG, 16(2), p66-67 and Krumlauf, 1994, Cell, 78(2), p 191-201). The HOX proteins are transcription factors that act to define anterior-posterior development in early development. Such PBX or HOX genes or proteins as described herein include homologues present in any multicellular animal, but are suitably from vertebrates, e.g. from mammals, in particular from humans.

As referred to herein “binding” refers to the interaction or association of at least two moieties in a reversible or irreversible reaction, wherein said binding is suitably specific and selective.

As used herein a “binding partner” refers to a molecule which recognizes and binds specifically (i.e. in preference to binding to other molecules) to its binding partner. Such binding pairs when bound together form a complex.

A “reduction in binding” refers to a decrease in binding, e.g. as manifest by an increased concentration of one of the binding pair required to achieve binding. Reduction includes a slight decrease as well as absolute abrogation of specific binding. A total reduction of specific binding is considered to equate to a prevention of binding.

“Inhibition” refers to competitive interference of the binding of the binding partners by the compound, which serves to reduce the partners' binding.

Agents which prevent or reduce PBX-dependent transcription regulation, have been found to have advantageous effects on aberrant cell division (International patent application PCT/GB2003/005425 published as WO2004/055049 1 Jul. 2004 and International patent application PCT/GB2006/002390 published as WO2007/00601 4 January 2007, the contents of which are incorporated in their entirety for all purposes).

Such agents are typically those which prevent, reduce or inhibit the binding of PBX to its binding partners, especially preferably the binding between PBX and HOX (such as antagonists of the interaction between HOX and PBX, e.g. the compounds of invention). However, suitable agents also include those that affect binding of the transcription factors to the target DNA, e.g. which block the interaction of PBX or its binding partner, such as HOX, to the target DNA. Suitably, such agents prevent HOX-dependent transcription regulation.

Whilst not wishing to be bound by theory, it is believed that antagonists of HOX:PBX binding prevent the interaction between multiple important HOX:PBX protein binding partners, and the HOX proteins are therefore unable to act as transcription factors on the genes to which they bind. The failure to regulate expression of these genes may have numerous effects on the cells, for example reducing or preventing the excessive cell division and inducing cell death. Similarly, any moiety which prevents or reduces PBX-dependent transcription regulation, e.g. blocks the interaction of HOX with its target DNA, may be expected to have similar effects.

Agents which are suitable for this purpose include antagonists of the interaction between HOX and/or PBX and the DNA target to which they bind, antagonists of the interaction between PBX and its binding partners, typically HOX proteins, or agents which impair the binding ability of HOX/PBX or the target DNA, e.g. which block relevant sites or cause structural changes at relevant sites on HOX/PBX or the target DNA or reduce the number of molecules available for binding (which may be achieved by for example modifying the expression/expressed product of PBX/HOX). Suitably however, antagonists are employed. Suitable agents are the compounds of the invention as described above.

In a further aspect, therefore, the present invention provides a method of reducing aberrant cell division wherein said cells are administered a compound of the invention, hereinafter alternately referred to as “agent of the invention” which prevents or reduces PBX-dependent transcription regulation, suitably which reduces or prevents binding of PBX to a binding partner, preferably to HOX (suitably HOXB4, HOXB8 or HOXA9) or reduces or prevents binding of HOX to its target DNA, suitably an antagonist, suitably an antagonist of the interaction between HOX and PBX, and which suitably inhibits HOX-dependent transcription regulation.

As described herein, “aberrant cell division” refers to cell division above the normal level (i.e. abnormal cell division) considered appropriate under the conditions which exist. Markers of aberrant cell division are well known to the person skilled in the art and can be used to determine whether a particular cell has been effected. For example, cells undergoing aberrant cell division may show atypical cytology, for example cellular pleomorphism, nuclear pleomorphism, nuclear hyperchromatism or an increased nuclear cytoplasmic ratio. Cells undergoing aberrant cell division may show a failure of cell differentiation. More particularly, such aberrant cell division may be present in certain conditions or diseases/disorders as described hereinafter, such as a cancer.

“Reducing” cell division refers to reducing the rate of cell growth. Suitably, cell growth is reduced to less than 0.5, in particular less than 0.25, e.g. less than 0.1 relative to control growth (without the agent) over the same time period (wherein control growth=1). Suitably, reduced cell division encompasses cell death/lack of viability which may occur in addition, or as an alternative to the reduction in cell growth. When cell death occurs suitably more than 50% of the existing cells, in particular more than 75% of the cells, are destroyed.

By adjusting the dose of the agent used it may also be possible to completely ablate some malignancies. Compounds of the invention may therefore be used to slow the growth of, or completely destroy, cancerous cells. As explained in more detail below, a suitable dose will depend on a number of factors and can be determined by a skilled practitioner.

As described herein “PBX-dependent transcription regulation” refers to activation or suppression of the transcription of genes by processes in which PBX plays a pivotal role, e.g. acts as a cofactor in the transcription regulatory complexes.

Prevention or reduction refers to a measurable change in the extent of transcription.

Prevention equates to a reduction in transcription to undetectable levels.

“Target DNA” refers to the gene containing the regulatory region to which PBX, HOX or any member of the transcription regulation complex containing such proteins, binds.

As referred to herein, an “antagonist” is a molecule or complex of molecules which by virtue of structural similarity to one molecule of a binding pair competes with that molecule for binding to the other molecule of the binding pair.

As specifically referred to herein, the antagonists of the invention are antagonists of the interaction between HOX and PBX which prevent or reduce binding between those entities. Suitable antagonists bind to, or compete with the binding site on HOX or PBX. Typically antagonists compete by mimicking the PBX binding site on HOX, i.e. binding to PBX.

Such methods may be performed in vitro, in vivo or ex vivo.

Having regard to their ability to specifically block the interaction between PBX and HOX and inhibit aberrant cellular division, the compounds of the invention, hereinafter alternately referred to as “agents of the invention”, are useful in the treatment or prevention of a condition or disorder in which aberrant cell division occurs, particularly a cancer

Treatment in accordance with the invention may be symptomatic or prophylactic,

Thus in a further aspect the invention includes an agent of the invention for use as a pharmaceutical.

Therefore according to a further aspect, the invention provides an agent of the invention for treating or preventing a condition or disorder in which aberrant cell division occurs.

Therefore according to a further aspect, the invention provides the use of an agent of the invention in the manufacture of a medicament for the prevention or treatment of a condition or disorder in which aberrant cell division occurs.

Therefore according to a further aspect, the invention provides a method for preventing or treating a condition or disorder in which aberrant cell division which comprises administering to a subject in need thereof a therapeutically effective amount of an agent of the invention.

In accordance with the foregoing, the invention also provides as a further aspect a method for preventing or treating a condition or disorder in which aberrant cell division occurs, particularly cancer, which comprises administering to a subject, particularly a human subject, in need thereof a therapeutically effective amount of an agent of the invention.

In another aspect the invention provides an agent of the invention for preventing or treating a condition or disorder in which aberrant cell division occurs, particularly cancer.

In another aspect the invention provides the use of an agent of the invention in the manufacture of a medicament for the prevention or treatment of a condition or disorder in which aberrant cell division occurs, particularly cancer.

As referred to herein a “disorder” or a “disease” refers to an underlying pathological disturbance in a symptomatic or asymptomatic organism relative to a normal organism, which may result, for example, from infection or an acquired or congenital genetic imperfection.

A “condition” refers to a state of the mind or body of an organism which has not occurred through disease, e.g. the presence of a moiety in the body such as a toxin, drug or pollutant.

As used herein, the term “treat”, “treating” or “treatment” of any disease or disorder refers in one embodiment, to ameliorating the disease or disorder (i.e., slowing or arresting or reducing the development of the disease or at least one of the clinical symptoms thereof). In another embodiment “treat”, “treating” or “treatment” refers to alleviating or ameliorating at least one physical parameter including those which may not be discernible by the patient. In yet another embodiment, “treat”, “treating” or “treatment” refers to modulating the disease or disorder, either physically, (e.g., stabilization of a discernible symptom), physiologically, (e.g., stabilization of a physical parameter), or both. In yet another embodiment, “treat”, “treating” or “treatment” refers to preventing or delaying the onset or development or progression of the disease or disorder. For example, symptoms which may be affected include tumour size or numbers of cancerous cells in a given sample (or reduced stem cell numbers as described hereinafter).

“Prevention” of a condition or disorder refers to delaying or preventing the onset of a condition or disorder or reducing its severity, as assessed by the appearance or extent of one or more symptoms of said condition or disorder.

As used herein, the term “subject” refers to an animal. Typically the animal is a mammal. A subject also refers to for example, primates (e.g., humans), cows, sheep, goats, horses, dogs, cats, rabbits, rats, mice, fish, birds and the like. In certain embodiments, the subject is a primate. In yet other embodiments, the subject is a human.

As used herein, a subject is “in need of” a treatment if such subject would benefit biologically, medically or in quality of life from such treatment.

The term “a therapeutically effective amount” of an agent of the invention refers to an amount of the agent of the invention that will elicit the biological or medical response of a subject, for example, reduction or inhibition of an enzyme or a protein activity, or ameliorate symptoms, alleviate conditions, slow or delay disease progression, or prevent a disease, etc. In one non-limiting embodiment, the term “a therapeutically effective amount” refers to the amount of the agent of the invention that, when administered to a subject, is effective to at least partially alleviating, inhibiting, preventing and/or ameliorating a condition or disorder in which aberrant cell division occurs. In another non-limiting embodiment, the term “a therapeutically effective amount” refers to the amount of the agent of the invention that, when administered to a cell, or a tissue, or a non-cellular biological material, or a medium, is effective to at least partially reducing aberrant cell division.

As an alternative to performing the methods in vivo, such methods may be performed in vitro, e.g. to reduce the cell division of, or eliminate, cells undergoing aberrant cell growth, in a sample. Appropriate culture conditions are as described for other methods of the invention as described hereinafter.

This is particularly useful in cell samples containing both normal and aberrant cells in which aberrant cells may be controlled/removed and the sample containing the normal cells used for subsequent procedures, e.g. returned to the donor body. This may be useful to, for example, eliminate aberrant haematopoietic blood cells from a blood sample of a patient, e.g. leukaemic cells, and the remaining cells may then be returned to the body of that patient.

Thus in a yet further aspect the present invention provides a method of reducing aberrant cell division (in particular of reducing the growth, more particularly involving the death and hence reducing the number, of cancer cells) in cells in a sample, wherein an agent of the invention as described hereinbefore is administered to said sample. In a method for treating patients suffering from a disorder or condition typified by aberrant cell division (or preventing the same), said sample may be harvested from said patient and then returned to that patient as described hereinafter. In this context, a “sample” refers to any material obtained from a human or non-human animal, including embryonic, foetal, immature and adult stages of said animal, which contains cells undergoing aberrant cell division and include tissues and body fluids.

“Body fluids” in this case include in particular blood, spinal fluid and lymph and “tissues” include tissue obtained by surgery or other means.

Suitably, the aberrant cell division occurs in cells from eukaryotic organisms which may be any eukaryotic organisms such as human beings, other mammals and animals, birds, insects and fish.

Non-human animals from which cells may be derived or on which methods of the invention maybe conducted include, but are not limited to mammals, particularly primates, domestic animals, livestock and laboratory animals. Thus animals include mice, rats, chickens, frogs, guinea pigs, cats, dogs, pigs, cows, goats, sheep, horses. Suitably, the cells are derived from, and the methods used to treat, or be prophylactic in, humans.

In particular, the cells undergoing aberrant cell division are cancer cells and the disorder to be treated or prevented is a cancer. Cancers that can be treated in this way are those cancers which involve the expression of HOX and PBX genes, wherein HOX/PBX dimer expression is reduced by the activity of a compound of the invention, thus blocking the growth of, reducing the proliferation of, or leading directly to the death of, the cancerous cells.

In a further embodiment, the compound of the invention may act on the cancerous cells to move them from a quiescent state into the cell cycle and thus make them more susceptible to other, e.g. cytotoxic, anti-cancer treatments.

Suitably said cell to be treated expresses one or more Hox genes. For example, said cell may express one or more of HOXA1, HOXA3, HOXA4, HOXA5, HOXA7, HOXA9, HOXB1, HOXB2, HOXB3, HOXB4, HOXB8, HOXB9, HOXC4, HOXC6, HOXC8, HOXD3, HOXD4, HOXD8, HOXD9. Said cell may express one or more of HOXB4, HOXB8 and HOXA9. It is possible that the level of Hox gene expression in the cell may be directly related to the sensitivity of the cell to the compounds of the invention. The compounds of the invention would therefore be more effective at treating cells which show high levels of HOX gene expression, for example higher levels of HOX gene expression than that in the surrounding tissue or higher levels of HOX gene expression than that of other cancer types where the cell is a cancer cell. The methods of the invention may therefore be particularly suitable where the cells to be treated show such increased or higher levels of HOX gene expression.

Suitably said cancers are malignant or pre-malignant or benign tumours and include carcinomas, sarcomas, gliomas, melanomas and lymphomas, including cancers of the bladder, kidney, pancreas, brain, head and neck, breast, gut, prostate, lung and ovary and leukaemias and lymphomas. In particular, colorectal, pancreatic, bladder, prostate, cervical, ovarian, gastric and non-small cell lung cancers.

A condition or disorder characterised by aberrant cell division is a cancer, including, but not limited to, mesothelioma, hepatobilliary (hepatic and billiary duct), a primary or secondary CNS tumor, a primary or secondary brain tumor, lung cancer (NSCLC and SCLC), bone cancer, pancreatic cancer, melanoma and non-melanomatous skin cancer, cancer of the head or neck, cutaneous or intraocular melanoma, ovarian cancer, colon cancer, rectal cancer, cancer of the anal region, stomach cancer, gastrointestinal (gastric, colorectal, and duodenal), gastrointestinal stromal tumor, breast cancer, uterine cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cartilidge, or bone, cancer of the urethra, cancer of the penis, prostate cancer, testicular cancer, testicular lymphoma, chronic or acute leukemia, chronic myeloid leukemia, lymphocytic lymphomas, cancer of the bladder, cancer of the kidney or ureter, renal cell carcinoma, carcinoma of the renal pelvis, neoplasms of the central nervous system (CNS), primary CNS lymphoma, non hodgkins's lymphoma, spinal axis tumors, brain stem glioma, pituitary adenoma, adrenocortical cancer, gall bladder cancer, multiple myeloma, cholangiocarcinoma, fibrosarcoma, neuroblastoma, retinoblastoma, or a combination of one or more of the foregoing cancers.

In one embodiment of the present invention the cancer is lung cancer (NSCLC and SCLC), melanoma, cancer of the head or neck, ovarian cancer, colon cancer, rectal cancer, cancer of the anal region, stomach cancer, breast cancer, cancer of the kidney or ureter, renal cell carcinoma, carcinoma of the renal pelvis, cancer of the thyroid gland, cancer of the parathyroid gland, pancreatic cancer, prostate cancer, neoplasms of the central nervous system (CNS), primary CNS lymphoma, non hodgkins's lymphoma, or spinal axis tumors, or a combination of one or more of the foregoing cancers.

In a particular embodiment, the cancer is lung cancer (NSCLC and SCLC), melanoma, cancer of the head or neck, ovarian cancer, breast cancer, prostate cancer, colon cancer, or renal cell carcinoma.

In another embodiment, said condition or disorder in which aberrant cell division occurs is a benign proliferative disease, including, but not limited to, psoriasis, benign prostatic hypertrophy or restinosis.

In one embodiment, said condition or disorder in which aberrant cell division occurs is myelodysplasia (MDS).

In some cancers, for example some forms of human pre-B cell leukaemia, PBX may act as an oncogene. The effects of PBX in such cancers will be different to that in other cancer types where PBX is not an oncogene. The effects of a compound of the invention may also therefore be different.

In one embodiment, therefore, the present invention does not apply to such cancers because the effect of a compound of the invention will be via a different mechanism to the PBX:HOX effect described hereinabove. In this embodiment, therefore, a compound of the invention may be used in the treatment or prevention of a cancer or other disorder in which aberrant cell division occurs, and in which PBX does not act as an oncogene. Suitably, the cancerous cells express one or more Hox genes. For example, a suitable cancer for treatment by a method of the invention may be a leukaemia other than human pre-B cell leukaemia.

In some cancers, such as acute myeloid leukaemia (AML), compounds of the invention may block the proliferation of the cancerous cells, but may also stimulate those cells to leave the GO/G 1 quiescent state and enter the cell cycle. These two effects are seen in the same cells under the same conditions. This is likely to be due to the cells being triggered to leave GO/G 1 by the compound (i.e. enter the cell cycle) but then failing to divide and instead either differentiating or undergoing apoptosis. The compounds of the invention may be used in the treatment of both primary AML and mature myeloid leukaemias. This suggests a specific utility for the compounds of the invention in acute myeloid and lymphoid leukaemias. Blocking PBX/HOX interactions in these cells using a compound of the invention may therefore form an effective treatment for preventing leukaemia cell growth in vivo. In addition, by increasing the proportion of leukaemic cells that enter the cell cycle, the compounds of the invention may also increase their sensitivity to other cancer treatments such as chemotherapy. The compounds of the invention may therefore be used in combination with another cancer treatment as described further below.

Agents which prevent or reduce PBX-dependent transcription regulation have also been found to have beneficial effects on stem cells.

“Stem cells” as referred to herein are undifferentiated cells which are capable of differentiating into various cells, e.g. various blood cell types, and include haematopoietic (e.g. found in the bone marrow) and neural and hepatic stem cells, embryonic stem cells and embryonic germ cells and encompass both pluri- and toti-potent cells. Embryonic cells are considered to be those cells derived from the inner cell mass of the blastocyst and embryonic germ cells are those cells isolated from the primordial germ cell of the gonadal ridge of the 5 to 10 week old foetus. Suitably, the cells are derived from eukaryotic organisms as described previously.

Prevention of PBX-mediated transcription regulation results in reduced, but continued, cell division and the appearance of molecular markers of differentiation (e.g. CD38). However on removal of the agent blocking that transcriptional regulation, cells reverted to stem cells as assessed by the appearance of molecular markers (e.g. HOXB4, HOXB8, HOXA9, AC133), thus reflecting pluripotency of the cells (International patent application PCT/GB2006/002390 published as WO2007/00601 4 Jan. 2007, the contents of which are incorporated in their entirety for all purposes). Whilst not wishing to be bound by theory, it is believed that despite the appearance of markers of differentiation/maturation, no phenotypic changes symptomatic of differentiation occur and the cells instead have a significantly reduced rate of cell cycling while the agent is being administered. On removal of the agent, the cells reverted to stem cells.

It is also believed that treatment of pluripotent haematopoietic stem and progenitor cells (HSPCs) with a compound of the invention may block their proliferation, and increase the proportion of cells in the GO-G1 phase of the cell cycle. The longevity of the cultures confirms the effects of putative stem cells as well as more differentiated progenitor populations. The specificity of this inhibitory effect on these gene targets is underlined by its reversibility, with gene transcription and cell growth resuming on removal of the agent.

These results have a number of applications which include maintenance or expansion of stem cells (e.g. in culture), for example for temporary storage of said cells, with possible expansion during that storage period. Such cells may then, for example, be used in clinical applications in which the addition of stem cells is desirable, e.g. to patients that have reduced numbers of stem cells and/or the ability to produce certain differentiated cell types, due to, for example, age, disease (e.g. cancers or autoimmune disease), congenital factors, environmental influences or contaminants and/or administered chemicals. In particular stem cells may be harvested from a patient prior to chemotherapy or radiotherapy and maintained and/or expanded and returned to that patient after chemotherapy or radiotherapy.

As an alternative example, the stem cells may be used to provide cells from which a particular differentiated cell may be formed, e.g. neuronal cells, particularly in adult recipients where such suitable stem cells are absent or only low levels are present. The recipient of the stem cells is suitably also the donor, but may also be a different individual. The compounds of the invention may therefore be used to protect explanted tissue that contains stem cells (e.g. bone marrow cells) during culture in vitro or ex vivo.

Cells may also be maintained ex vivo or in vivo, for example to maintain viability during treatment that might normally affect their viability, e.g. during chemo- or radio-therapy.

Agents as described herein, i.e. compounds of the invention, can be used to reduce the susceptibility of stem cells to damage by such treatments by temporarily stopping or slowing the cell cycle of the stem cells. For example, compounds of the invention may be used to reduce the side effects caused by other cancer treatments, e.g. cytotoxic shock associated with many chemotherapeutic regimes. The cytoprotective effect of compounds of the invention on stem cells in vivo may also allow higher levels or doses of such cancer treatments to be used due to the decreased side-effects produced. For example, a higher dose of chemo-or radio-therapy may be possible.

Thus in a further aspect, the present invention provides a method of maintaining or expanding stem cells, wherein said method comprises at least the step of contacting said cells with an agent of the invention as described hereinbefore, suitably an antagonist, suitably an antagonist of the interaction between HOX and PBX. This method may be used to maintain pluri- or toti-potency of the stem cells.

Suitably this method is performed in vitro or ex vivo, in culture, in which case the method may contain an initial step of harvesting stem cells from a donor. However, the method may also be used in vivo to maintain or improve the numbers of stem cells in an individual, particularly during exposure to agents or treatments that might cause stem cell damage. In such circumstances, the present invention provides a method of maintaining or expanding stems cells in a patient wherein said patient is administered an agent of the invention, suitably an antagonist, suitably an antagonist of the interaction between HOX and PBX.

“Maintaining” the cells refers to maintaining the viability of a large proportion of the starting, e.g. harvested, cells with minimal cell division, during the course of the treatment or culture period.

“Expanding” the cells refers to at least some cell division, suitably significant cell division, to increase the numbers of cells during the course of treatment, or culture.

As referred to herein “culture” refers to the growth or maintenance of the cells in a controlled artificial environment, i.e. ex vivo. Standard techniques for culture of cells are well known. Suitably cells are cultured at 37° C., 5% CO₂ in a humidified atmosphere in a standard culture medium. Suitably said culture is conducted for at least 2 hours, suitably more than 24 hours; e.g. between 24 hours and 8 weeks.

“Contacting” as used herein refers to any suitable technique which allows the agent to have access, and thus the possibility of binding, to cells in the sample, e.g. by application to the culture medium.

After the cells have been maintained or expanded, the agent may be removed to recover pluri- or toti-potency. When the method is performed in vivo this may be achieved by ceasing administration and allowing the body to clear the agent. In vitro or ex vivo, the agent is removed from the culture medium, e.g. by washing and replacement with fresh medium. Alternatively, the agent may be removed by allowing it to degrade naturally.

Thus the invention provides a method of maintaining or expanding stem cells and/or obtaining pluri- or toti-potent stem cells, in culture, suitably an expanded population of said cells, wherein said method comprises at least the steps of:

a) contacting said cells in culture with an agent of the invention, which reduces or prevents PBX dependent transcription regulation as described hereinabove, suitably an antagonist, suitably an antagonist of the interaction between HOX and PBX; b) culturing said cells in the absence of said agent.

It should be noted that the peptide becomes degraded within a few days during culture and thus active peptide is depleted. Thus, step b) may be performed without any prior washing if sufficient time has lapsed for degradation to occur. As mentioned previously, culture times are at least 2 hours, suitably more than 24 hours, e.g. between 24 hours and 8 weeks.

The method may contain an initial step of harvesting stem cells from a donor.

Cells obtained by this and other methods of the invention comprise further aspects of the invention as does their use as a medicament.

The cells thus prepared by the above described in vitro or ex vivo methods may then be administered to an individual in need of such stem cells. Optionally, the cells may be modified prior to transplant, e.g. during the course of culturing or just prior to transplanting, e.g. by genetic modification, e.g. for gene transfer or to import a function not previously present in said cells, e.g. to compensate for a genetic deficit, e.g. by providing a missing factor, e.g. adenosine deaminase (ADA).

Thus in a yet further aspect, the present invention provides a method of treating an individual in need of stem cells wherein stem cells prepared according to the above described method are administered to said individual.

Suitably said individual in need of said stem cells is an individual who has (or will have) lower than normal or desirable levels of such cells, which condition may exist normally, e.g. through age or as a result of external factors e.g. through chemotherapy or radiotherapy. Suitably, said stem cells are derived from the recipient individual.

Thus, the present invention provides a method of improving the number of stem cells in a recipient individual wherein said method comprises at least the steps of:

a) harvesting stem cells from a donor,
b) culturing said stem cells according to the methods described hereinabove;

c) administering said cultured stem cells to said recipient individual.

Suitably, the method is a method of improving the number of stem cells in a patient subject to chemotherapy or radiotherapy, wherein said method comprises at least the steps of:

a) harvesting stem cells from said patient prior to chemotherapy or radiotherapy, b) culturing said stem cells according to the methods described hereinbefore;

c) administering said cultured stem cells to said patient after completion of chemotherapy or radiotherapy.

Alternatively, harvesting step a) in the methods above may be absent and step b) may comprise culturing stem cells harvested from the donor according to the methods described hereinbefore. Said cells may be harvested by obtaining a sample of cells, tissue or body fluid from said donor and optionally extracting the cells therefrom.

As used herein a “sample” refers to any material obtained from the donor, e.g. human or non-human animal, including embryonic, foetal, immature and adult stages of said animal, which contains stem cells and includes, tissues and body fluids.

“Body fluids” include blood and spinal fluid.

“Tissue samples” include tissue obtained by surgical interventions (e.g. bone marrow or liver) or by other means e.g. placenta and umbilical cord. The animals from which cells are derived or to which the methods are applied are preferably as described’ hereinabove in connection with the methods of—reducing aberrant cell division.

As used herein reference to “improving the number of stem cells” refers to increasing the number of stem cells to be added (suitably of the particular type to be added, e.g. haematopoietic stem cells) relative to the number present in the individual at the time at which administration would occur. Thus in the case of a patient subject to chemotherapy or radiotherapy the observed improvement is in the number of stem cells in a patient post-chemotherapy or post-radiotherapy. An improvement may also consist of the addition of certain stem cells previously absent or present in very low numbers, e.g. neuronal stem cells.

Alternatively, the present invention provides an agent of the invention for the treatment or prevention of conditions or disorders typified by a need for stem cells, suitably in treating or preventing conditions or disorders in which stem cell numbers are lower than normal, e.g. due to chemotherapy or radiotherapy, or in conditions in which the provision of stem cells may allow the production of one or more particular differentiated cells that are absent or present in abnormally low numbers, or lower numbers than desired, at the site of interest.

Conditions or disorders in which stem cell numbers are lower than normal include autoimmune disorders, radiotherapy, chemotherapy and certain viral infections.

Conditions in which the use of stem cells by transplantation may provide appropriate differentiated cells which are absent or present at lower than normal or lower than desired levels include Alzheimer's disease, Parkinson's disease and other age-related disorders or conditions (including cosmetic treatments), multiple sclerosis, spinal cord injury, diabetes, chronic heart disease, end-stage kidney disease, liver failure and in which stem cells are used to replace destroyed or dysfunctional cells. Prevention of such conditions or disorders may be achieved by maintaining stem cells in a protected state by the use of an agent of the invention.

The present invention further provides cells prepared by the methods described hereinabove for the treatment of conditions or disorders typified by a need for stem cells, as described above.

It should be noted that due to the effects of the aforementioned agents on aberrant cell division, even samples of stem cells containing such aberrant cells may be used and a dual effect of reducing the aberrant division while expanding the stem cells may be achieved. Thus the aforementioned agents may be used in vitro, ex vivo or in vivo to protect normal stem/progenitor cells whilst eliminating cells undergoing aberrant cell growth. This is particularly applicable to haematopoietic cells, e.g. when treating leukaemia/lymphoma.

Thus in a particular aspect the present invention provides a method of treating or preventing a condition or disorder in which aberrant cell division occurs. e.g., a cancer, in a human or non-human subject, wherein said method comprises administering an agent of the invention, wherein said agent is capable of both reducing said aberrant cell division and maintaining or expanding stem cells of said subject.

As described above, the agents of the invention, which reduce or prevent PBX-dependent transcription regulation, particularly HOX:PBX antagonists, have various clinical applications and thus a further aspect of the invention provides pharmaceutical compositions containing agents of the invention. The use of these agents as a medicament forms a further aspect of the invention.

Thus, in a further aspect the present invention provides a pharmaceutical composition comprising an agent of the invention, which reduces or prevents PBX-dependent transcription regulation as described hereinabove, suitably an antagonist, suitably an antagonist of the interaction between HOX and PBX, or a polynucleotide or vector capable of expressing such a peptide, and a pharmaceutically acceptable carrier.

Pharmaceutical compositions as described herein for use as a medicament, in particular for use in treating or preventing disorders or conditions typified by aberrant cell division, or disorders or conditions typified by a need for stem cells, such as the conditions described herein, and methods of treatment or prophylaxis using such compositions and use of said agents for the preparation of a medicament for treating or preventing such disorders or conditions, form further aspects of the invention.

“Pharmaceutically acceptable” as referred to herein refers to ingredients that are compatible with other ingredients of the compositions as well as physiologically acceptable to the recipient.

As used herein, the term “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, binders, excipients, disintegration agents, lubricants, sweetening agents, flavoring agents, dyes, and the like and combinations thereof, as would be known to those skilled in the art (see, for example, Remington's Pharmaceutical Sciences, 18th Ed. Mack Printing Company, 1990, pp. 1289-1329). Except insofar as any conventional carrier is incompatible with the active ingredient, its use in the therapeutic or pharmaceutical compositions is contemplated.

Pharmaceutical compositions according to the invention may be formulated in conventional manner using readily available ingredients. Thus, the active ingredient (i.e. the peptide) may be incorporated, optionally together with other active substances, with one or more conventional carriers, diluents and/or excipients, to produce conventional galenic preparations such as tablets, pills, powders, lozenges, sachets, cachets, elixirs, suspensions, emulsions, solutions, syrups, aerosols (as a solid or in a liquid medium), ointments, soft and hard gelatin capsules, suppositories, sterile injectable solutions, sterile packaged powders, and the like.

The pharmaceutical composition can be formulated for particular routes of administration such as oral administration, parenteral administration, and rectal administration, etc. In addition, the pharmaceutical compositions of the present invention can be made up in a solid form (including without limitation capsules, tablets, pills, granules, powders or suppositories), or in a liquid form (including without limitation solutions, suspensions or emulsions). The pharmaceutical compositions can be subjected to conventional pharmaceutical operations such as sterilization and/or can contain conventional inert diluents, lubricating agents, or buffering agents, as well as adjuvants, such as preservatives, stabilizers, wetting agents, emulsifers and buffers, etc.

Typically, the pharmaceutical compositions are tablets or gelatin capsules comprising the active ingredient together with

a) diluents, e.g., lactose, polylactone, dextrose, sucrose, mannitol, sorbitol, cellulose and/or glycine;
b) lubricants, e.g., silica, talcum, stearic acid, its magnesium or calcium salt and/or polyethylene glycol; for tablets also
c) binders, e.g., magnesium aluminum silicate, starch paste, gelatin, tragacanth, methylcellulose, sodium carboxymethylcellulose and/or polyvinylpyrrolidone; if desired
d) disintegrants, e.g., starches, agar, alginic acid or its sodium salt, or effervescent mixtures; and/or
e) absorbents, colorants, flavors and sweeteners.

Tablets may be either film coated or enteric coated according to methods known in the art.

Suitable compositions for oral administration include an effective amount of an agent of the invention in the form of tablets, lozenges, aqueous or oily suspensions, dispersible powders or granules, emulsion, hard or soft capsules, or syrups or elixirs. Compositions intended for oral use are prepared according to any method known in the art for the manufacture of pharmaceutical compositions and such compositions can contain one or more agents selected from the group consisting of sweetening agents, flavoring agents, coloring agents and preserving agents in order to provide pharmaceutically elegant and palatable preparations. Tablets may contain the active ingredient in admixture with nontoxic pharmaceutically acceptable excipients which are suitable for the manufacture of tablets. These excipients are, for example, inert diluents, such as calcium carbonate, sodium carbonate, lactose, calcium phosphate or sodium phosphate; granulating and disintegrating agents, for example, corn starch, or alginic acid; binding agents, for example, starch, gelatin or acacia; and lubricating agents, for example magnesium stearate, stearic acid or talc. The tablets are uncoated or coated by known techniques to delay disintegration and absorption in the gastrointestinal tract and thereby provide a sustained action over a longer period. For example, a time delay material such as glyceryl monostearate or glyceryl distearate can be employed. Formulations for oral use can be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example, peanut oil, liquid paraffin or olive oil.

Certain injectable compositions are aqueous isotonic solutions or suspensions, and suppositories are advantageously prepared from fatty emulsions or suspensions. Said compositions may be sterilized and/or contain adjuvants, such as preserving, stabilizing, wetting or emulsifying agents, solution promoters, salts for regulating the osmotic pressure and/or buffers. In addition, they may also contain other therapeutically valuable substances. Said compositions are prepared according to conventional mixing, granulating or coating methods, respectively, and contain about 0.1-75%, or contain about 1-50%, of the active ingredient.

Suitable compositions for transdermal application include an effective amount of an agent of the invention with a suitable carrier. Carriers suitable for transdermal delivery include absorbable pharmacologically acceptable solvents to assist passage through the skin of the host. For example, transdermal devices are in the form of a bandage comprising a backing member, a reservoir containing the compound optionally with carriers, optionally a rate controlling barrier to deliver the compound of the skin of the host at a controlled and predetermined rate over a prolonged period of time, and means to secure the device to the skin.

Suitable compositions for topical application, e.g., to the skin and eyes, include aqueous solutions, suspensions, ointments, creams, gels or sprayable formulations, e.g., for delivery by aerosol or the like. Such topical delivery systems will in particular be appropriate for dermal application, e.g., for the treatment of skin cancer, e.g., for prophylactic use in sun creams, lotions, sprays and the like. They are thus particularly suited for use in topical, including cosmetic, formulations well-known in the art. Such may contain solubilizers, stabilizers, tonicity enhancing agents, buffers and preservatives.

As used herein a topical application may also pertain to an inhalation or to an intranasal application. They may be conveniently delivered in the form of a dry powder (either alone, as a mixture, for example a dry blend with lactose, or a mixed component particle, for example with phospholipids) from a dry powder inhaler or an aerosol spray presentation from a pressurised container, pump, spray, atomizer or nebuliser, with or without the use of a suitable propellant.

Dosages of agents of the invention employed in practising the present invention will of course vary depending, for example, on the particular condition to be treated, the effect desired and the mode of administration. In general, suitable daily dosages for administration by inhalation are of the order of 0.0001 to 30 mg/kg, typically 0.01 to 10 mg per patient, while for oral administration suitable daily doses are of the order of 0.01 to 100 mg/kg.

The present invention further provides anhydrous pharmaceutical compositions and dosage forms comprising the agents of the invention as active ingredients, since water may facilitate the degradation of certain compounds.

Anhydrous pharmaceutical compositions and dosage forms of the invention can be prepared using anhydrous or low moisture containing ingredients and low moisture or low humidity conditions. An anhydrous pharmaceutical composition may be prepared and stored such that its anhydrous nature is maintained. Accordingly, anhydrous compositions are packaged using materials known to prevent exposure to water such that they can be included in suitable formulary kits. Examples of suitable packaging include, but are not limited to, hermetically sealed foils, plastics, unit dose containers (e.g., vials), blister packs, and strip packs.

The invention further provides pharmaceutical compositions and dosage forms that comprise one or more agents that reduce the rate by which the compound of the present invention as an active ingredient will decompose. Such agents, which are referred to herein as “stabilizers,” include, but are not limited to, antioxidants such as ascorbic acid, pH buffers, or salt buffers, etc.

The agent of the invention may be administered either simultaneously with, or before or after, one or more other therapeutic agent. The agent of the invention may be administered separately, by the same or different route of administration, or together in the same pharmaceutical composition as the other agents.

In one embodiment, the invention provides a product comprising an agent of the invention and at least one other therapeutic agent as a combined preparation for simultaneous, separate or sequential use in therapy. In one embodiment, the therapy is the treatment of a condition or disorder in which aberrant cell division occurs. Products provided as a combined preparation include a composition comprising the agent of the invention and the other therapeutic agent(s) together in the same pharmaceutical composition, or the agent of the invention and the other therapeutic agent(s) in separate form, e.g. in the form of a kit.

In one embodiment, the invention provides a pharmaceutical composition comprising an agent of the invention and another therapeutic agent(s). Optionally, the pharmaceutical composition may comprise a pharmaceutically acceptable excipient, as described above.

A skilled person will appreciate that an agent of the invention may be administered to a subject, particularly a human subject, wherein the subject is being treated with surgery or radiotherapy for a condition or disorder in which aberrant cell division occurs. A compound of the invention may also be administered to a subject, particularly a human subject, wherein the subject has previously (e.g. within 24 hours) been treated with surgery or radiotherapy for a condition or disorder in which aberrant cell division occurs. A subject, particularly a human subject may also be treated with surgery or radiotherapy for a condition or disorder in which aberrant cell division occurs wherein a compound of the invention has previously (e.g. within 24 hours) been administered to a subject,

In one embodiment, the invention provides a kit comprising two or more separate pharmaceutical compositions, at least one of which contains an agent of the invention. In one embodiment, the kit comprises means for separately retaining said compositions, such as a container, divided bottle, or divided foil packet. An example of such a kit is a blister pack, as typically used for the packaging of tablets, capsules and the like.

The kit of the invention may be used for administering different dosage forms, for example, oral and parenteral, for administering the separate compositions at different dosage intervals, or for titrating the separate compositions against one another. To assist compliance, the kit of the invention typically comprises directions for administration.

In the combination therapies of the invention, the agent of the invention and the other therapeutic agent may be manufactured and/or formulated by the same or different manufacturers. Moreover, the agent of the invention and the other therapeutic may be brought together into a combination therapy: (i) prior to release of the combination product to physicians (e.g. in the case of a kit comprising the agent of the invention and the other therapeutic agent); (ii) by the physician themselves (or under the guidance of the physician) shortly before administration; (iii) in the patient themselves, e.g. during sequential administration of the agent of the invention and the other therapeutic agent.

Accordingly, the invention provides the use of an agent of the invention for treating a condition or disorder in which aberrant cell division occurs, wherein the medicament is prepared for administration with another therapeutic agent. The invention also provides the use of another therapeutic agent for treating a condition or disorder in which aberrant cell division occurs, wherein the medicament is administered with an agent of the invention.

The invention also provides an agent of the invention for use in a method of treating a condition or disorder in which aberrant cell division occurs, wherein the agent of the invention is prepared for administration with another therapeutic agent. The invention also provides another therapeutic agent for use in a method of treating a condition or disorder in which aberrant cell division occurs, wherein the other therapeutic agent is prepared for administration with an agent of the invention. The invention also provides an agent of the invention for use in a method of treating a condition or disorder in which aberrant cell division occurs, wherein agent of the invention is administered with another therapeutic agent. The invention also provides another therapeutic agent for use in a method of treating a condition or disorder in which aberrant cell division occurs, wherein the other therapeutic agent is administered with an agent of the invention.

The invention also provides the use of an agent of the invention for treating a condition or disorder in which aberrant cell division occurs, wherein the subject has previously (e.g. within 24 hours) been treated with another therapeutic agent. The invention also provides the use of another therapeutic agent for treating a condition or disorder in which aberrant cell division occurs, wherein the subject has previously (e.g. within 24 hours) been treated with an agent of the invention.

Compositions may additionally comprise molecules which assist or augment the action of the agents of the invention, e.g. cytotoxic agents such as antimetabolites, alkylating agents, cytotoxic antibiotics, topoisomerase I and/or II inhibitors, vinca alkaloids and monoclonal antibodies.

If required, the compositions may also contain targeting moieties attached to the active ingredient, e.g. a ligand which binds specifically and selectively to an endogenous receptor to allow targeting to a particular cell type or location, such as targeting to lymphocytes, monocytes, macrophages, endothelial cells, epithelial cells, blood cells, erythrocytes, platelets, eosinophils, neutrophils, natural killer cells, dendritic cells, brain cells, heart cells, lung cells, islet cells, kidney cells, cancer cells, hormonal gland cells, skin, bone, joints, bone marrow, gastric mucosa, lymph nodes, peyers patches, the omentum and other appropriate tissues.

Compounds of the invention may be used to assist or augment the action of agents used for conventional treatments, e.g. cytotoxic agents, to reduce their side effects, e.g. by protection of stem cells during treatment.

In one embodiment, a compound of the invention is administered alongside one or more other therapeutically active agents. For example, a compound of the invention may be used as a combinatorial chemotherapeutic agent. Compounds of the invention may induce some cancer cells, e.g. AML cells, to enter the cell cycle. Cells which have been stimulated in this way may therefore become more susceptible to conventional anti-cancer drugs. The componds of the invention may therefore be used in combination with other anti-cancer agents, such as cytotoxic drugs, to target cancers such as leukaemia, for example AML.

Compounds of the invention may also be used in combination with other anticancer therapies in order to protect the endogenous stem cell population. The compounds of the invention may maintain normal stem/progenitor cells in a GO/G1 quiescent state. This cytoprotective ability may thus protect such stem cells from the effects of any anti-cancer treatment. This may be of particular use where the compounds of the invention are used in combination with cytotoxic agents which target dividing cells. By maintaining the normal stem cells of the patient in a quiescent state during such treatment, the side effects of the anti-cancer treatment on the endogenous stem cell population can be minimised.

This reduction in the potential side effects may also allow a higher dose or level of the conventional treatment to be used on the patient than would otherwise be possible or safe.

In one embodiment, the other therapeutic agent is an anti-tumour agent selected from the group consisting of antiproliferative agents, kinase inhibitors, angiogenesis inhibitors, growth factor inhibitors, cox-I inhibitors, cox-II inhibitors, mitotic inhibitors, alkylating agents, antimetabolites, intercalating antibiotics, growth factor inhibitors, radiation, cell cycle inhibitors, enzymes, topoisomerase inhibitors, biological response modifiers, antibodies, cytotoxics, anti-hormones, statins, anti-androgens and photochemotherapy agents.

Accordingly, the invention includes as a further aspect a combination of an agent of the invention with an anti-tumour agent selected from the group consisting of antiproliferative agents, kinase inhibitors, angiogenesis inhibitors, growth factor inhibitors, cox-I inhibitors, cox-II inhibitors, mitotic inhibitors, alkylating agents, antimetabolites, intercalating antibiotics, growth factor inhibitors, radiation, cell cycle inhibitors, enzymes, topoisomerase inhibitors, biological response modifiers, antibodies, cytotoxics, anti-hormones, statins, anti-androgens and photochemotherapy agents.

In one embodiment of the present invention the anti-tumor agent used in conjunction with a composition of the present invention is an anti-angiogenesis agent, kinase inhibitor, pan kinase inhibitor or growth factor inhibitor.

Suitable pan kinase inhibitors include SU-11248 (sutinib malate), described in U.S. Pat. No. 6,573,293 (Pfizer Inc).

Anti-angiogenesis agents, include but are not limited to the following agents, such as EGF inhibitors, EGFR inhibitors, VEGF inhibitors, VEGFR inhibitors, TIE2 inhibitors, IGF1 R inhibitors, COX-II (cyclooxygenase II) inhibitors, MMP-2 (matrix-metalloprotienase 2) inhibitors, and MMP-9 (matrix-metalloprotienase 9) inhibitors. Suitable VEGF inhibitors, include for example, Avastin (bevacizumab), an anti-VEGF monoclonal antibody of Genentech, Inc. of South San Francisco, Calif.

Additional VEGF inhibitors include CP-547,632 (Pfizer Inc.), AG13736 (axitinib, Pfizer Inc.), ZD-6474 (AstraZeneca), AEE788 (Novartis), AZD-2171), VEGF Trap (Regeneron/Aventis), Vatalanib (also known as PTK-787, ZK-222584: Novartis & Schering AG), Macugen (pegaptanib octasodium, NX-1838, EYE-001, Pfizer Inc./Gilead/Eyetech), IM862 (Cytran Inc. of Kirkland, Wash., USA); and Angiozyme, a synthetic ribozyme from Ribozyme (Boulder, Colo.) and Chiron (Emeryville, Calif.) and combinations thereof. VEGF inhibitors useful in the practice of the present invention are disclosed in U.S. Pat. Nos. 6,534,524 and 6,235,764, both of which are incorporated in their entirety for all purposes. Particularly suitable VEGF inhibitors include CP-547,632, axitinib, Vatalanib, Macugen and combinations thereof.

Other antiproliferative agents that may be used with the compositions of the present invention include inhibitors of the enzyme farnesyl protein transferase and inhibitors of the receptor tyrosine kinase PDGFr. PDGRr inhibitors include but are not limited to those disclosed in international patent application publication number WO01/40217, published Jul. 7, 2001 and international patent application publication number WO2004/020431, published Mar. 11, 2004, the contents of which are incorporated in their entirety for all purposes. Suitable PDGFr inhibitors include Pfizer's CP-673,451 and CP-868,596 and its pharmaceutically acceptable salts.

Suitable GARF inhibitors include Pfizer's AG-2037 (pelitrexol and its pharmaceutically acceptable salts). GARF inhibitors useful in the practice of the present invention are disclosed in U.S. Pat. No. 5,608,082 which is incorporated in its entirety for all purposes.

Examples of useful COX-11 inhibitors which can be used in conjunction with compounds of the invention described herein include CELEBREX (celecoxib), parecoxib, deracoxib, ABT-963, MK-663 (etoricoxib), COX-189 (Lumiracoxib), BMS 347070, RS 57067, NS-398, Bextra (valdecoxib), paracoxib, Vioxx (rofecoxib), SD-8381, 4-Methyl-2-(3,4-dimethylphenyl)-1-(4-sulfamoyl-phenyl)-1 H-pyrrole, 2-(4-Ethoxyphenyl)-4-methyl-1-(4-sulfamoylphenyl)-1H-pyrrole, T-614, JTE-522, S-2474, SVT-2016, CT-3, SC-58125 and Arcoxia (etoricoxib). Additionally, COX-11 inhibitors are disclosed in U.S. patent application Ser. Nos. 10/801,446 and 10/801,429, the contents of which are incorporated in their entirety for all purposes

Other useful inhibitors as anti-tumor agents used in conjunction with compositions of the present invention include aspirin, and non-steroidal anti-inflammatory drugs (NSAIDs) which inhibit the enzyme that makes prostaglandins (cyclooxygenase I and II), resulting in lower levels of prostaglandins, include but are not limited to the following, Salsalate (Amigesic), Diflunisal (Dolobid), Ibuprofen (Motrin), Ketoprofen (Orudis), Nabumetone (Relafen), Piroxicam (Feldene), Naproxen (Aleve, Naprosyn), Diclofenac (Voltaren), Indomethacin (Indocin), Sulindac (Clinoril), Tolmetin (Tolectin), Etodolac (Lodine), Ketorolac (Toradol), Oxaprozin (Daypro) and combinations thereof.

Suitable COX-1 inhibitors include ibuprofen (Motrin), nuprin, naproxen (Aleve), indomethacin (Indocin), nabumetone (Relafen) and combinations thereof.

Targeted agents used in conjunction with a composition of the present invention include EGFr inhibitors such as Iressa (gefitinib, AstraZeneca), Tarceva (erlotinib or OSI-774, OSI Pharmaceuticals Inc.), Erbitux (cetuximab, Imclone Pharmaceuticals, Inc.), EMD-7200 (Merck AG), ABX-EGF (Amgen Inc. and Abgenix Inc.), HR3 (Cuban Government), IgA antibodies (University of Erlangen-Nuremberg), TP-38 (IVAX), EGFR fusion protein, EGF-vaccine, anti-EGFr immunoliposomes (Hermes Biosciences Inc.) and combinations thereof. Suitably EGFr inhibitors include Iressa, Erbitux, Tarceva and combinations thereof. Other anti-tumor agents include those selected from pan erb receptor inhibitors or ErbB2 receptor inhibitors, such as CP-724,714 (Pfizer, Inc.), CM 033 (canertinib, Pfizer, Inc.), Herceptin (trastuzumab, Genentech Inc.), Omitarg (2C4, pertuzumab, Genentech Inc.), TAK-165 (Takeda), GW-572016 (Ionafamib, GlaxoSmithKline), GW-282974 (GlaxoSmithKline), EKB-569 (Wyeth), PKM 66 (Novartis), dHER2 (HER2 Vaccine, Corixa and GlaxoSmithKline), APC8024 (HER2 Vaccine, Dendreon), anti-HER2/neu bispecific antibody (Decof Cancer Center), B7.her2.IgG3 (Agensys), AS HER2 (Research Institute for Rad Biology & Medicine), trifuntional bispecific antibodies (University of Munich) and mAB AR-209 (Aronex Pharmaceuticals Inc) and mAB 2B-1 (Chiron) and combinations thereof. Particular erb selective anti-tumor agents include Herceptin, TAK-165, CP-724,714, ABX-EGF, HER3 and combinations thereof. Suitably pan erb receptor inhibitors include GW572016, CM 033, EKB-569, and Omitarg and combinations thereof.

Additionally, other anti-tumor agents may be selected from the following agents, BAY-43-9006 (Onyx Pharmaceuticals Inc.), Genasense (augmerosen, Genta), Panitumumab (Abgenix/Amgen), Zevalin (Schering), Bexxar (Corixa/GlaxoSmithKline), Abarelix, Alimta, EPO 906 (Novartis), discodermolide (XAA-296), ABT-510 (Abbott), Neovastat (Aeterna), enzastaurin (Eli Lilly), Combrestatin A4P (Oxigene), ZD-6126 (AstraZeneca), flavopiridol (Aventis), CYC-202 (Cyclacel), AVE-8062 (Aventis), DMXAA (Roche/Antlsoma), Thymitaq (Eximias), Temodar (temozolomide, Schering Plough) and Revilimd (Celegene) and combinations thereof.

Other anti-tumor agents may be selected from the following agents, CyPat (cyproterone acetate), Histerelin (histrelin acetate), Plenaixis (abarelix depot), Atrasentan (ABT-627), Satraplatin (JM-216), thalomid (Thalidomide), Theratope, Temilifene (DPPE)1 ABI-007 (paclitaxel), Evista (raloxifene), Atamestane (Biomed-777), Xyotax (polyglutamate paclitaxel), Targetin (bexarotine) and combinations thereof.

Additionally, other anti-tumor agents may also be selected from the following agents, Trizaone (tirapazamine), Aposyn (exisulind), Nevastat (AE-941), Ceplene (histamine dihydrochloride), Orathecin (rubitecan), Virulizin, Gastrimmune (G17DT), DX-8951f (exatecan mesylate), Onconase (ranpimase), BEC2 (mitumoab), Xcytrin (motexafin gadolinium) and combinations thereof. Further anti-tumor agents may selected from the following agents, CeaVac (CEA), NeuTrexin (trimetresate glucuronate) and combinations thereof. Additional anti-tumor agents may selected from the following agents, OvaRex (oregovomab), Osidem (IDM-1), and combinations thereof.

Additional anti-tumor agents may selected from the following agents, Advexin (ING 201), Tirazone (tirapazamine), and combinations thereof. Additional anti-tumor agents may selected from the following agents, RSR13 (efaproxiral), Cotara (1311 chTNT 1/b), NBI-3001 (IL-4) and combinations thereof. Additional anti-tumor agents may selected from the following agents, Canvaxin, GMK vaccine, PEG Interon A, Taxoprexin (DHA/paciltaxel) and combinations thereof.

Other anti-tumor agents include Pfizer's MEK1/2 inhibitor PD325901, Array Biopharm's MEK inhibitor ARRY-142886, Bristol Myers' CDK2 inhibitor BMS-387,032, Pfizer's CDK inhibitor PD0332991 and AstraZeneca's AXD-5438 and combinations thereof.

Additionally, mTOR inhibitors may also be utilized such as CCI-779 (Wyeth) and rapamycin derivatives RAD001 (Novartis) and AP-23573 (Ariad), HDAC inhibitors SAHA (Merck Inc/Aton Pharmaceuticals) and combinations thereof. Additional anti-tumor agents include aurora 2 inhibitor VX-680 (Vertex), Chk1/2 inhibitor XL844 (Exilixis).

The following cytotoxic agents, e.g., one or more selected from the group consisting of epirubicin (Ellence), docetaxel (Taxotere), paclitaxel, Zinecard (dexrazoxane), rituximab (Rituxan), imatinib mesylate (Glivec), and combinations thereof, may be used in conjunction with a composition of the present invention as described herein.

The invention also contemplates the use of the compositions of the present invention together with hormonal therapy, including but not limited to, exemestane (Aromasin, Pfizer Inc.), leuprorelin (Lupron or Leuplin, TAP/Abbott/Takeda), anastrozole (Arimidex, Astrazeneca), gosrelin (Zoladex, AstraZeneca), doxercalciferol, fadrozole, formestane, tamoxifen citrate (tamoxifen, Nolvadex, AstraZeneca), Casodex (AstraZeneca), Abarelix (Praecis), Trelstar, and combinations thereof.

The invention also relates to hormonal therapy agents such as anti-estrogens including, but not limited to fulvestrant, toremifene, raloxifene, lasofoxifene, letrozole (Femara, Novartis), anti-androgens such as bicalutamide, flutamide, mifepristone, nilutamide, Casodex(R)(4′-cyano-3-(4-fluorophenylsulphonyl)-2-hydroxy-2-methyl-3′-(trifluoromethyl) propionanilide, bicalutamide) and combinations thereof.

Further, the invention provides a composition of the present invention alone or in combination with one or more supportive care products, e.g., a product selected from the group consisting of Filgrastim (Neupogen), ondansetron (Zofran), Fragmin, Procrit, Aloxi, Emend, or combinations thereof.

Particularly suitable cytotoxic agents include Camptosar, Erbitux, Iressa, Glivec, Taxotere and combinations thereof.

The following topoisomerase I inhibitors may be utilized as anti-tumor agents: camptothecin; irinotecan HCl (Camptosar); edotecarin; orathecin (Supergen); exatecan (Daiichi); BN-80915 (Roche); and combinations thereof. Particularly preferred toposimerase II inhibitors include epirubicin (Ellence).

Alkylating agents include, but are not limited to, nitrogen mustard N-oxide, cyclophosphamide, ifosfamide, melphalan, busulfan, mitobronitol, carboquone, thiotepa, ranimustine, nimustine, temozolomide, AMD-473, altretamine, AP-5280, apaziquone, brostallicin, bendamustine, carmustine, estramustine, fotemustine, glufosfamide, ifosfamide, KW-2170, mafosfamide, and mitolactol; platinum-coordinated alkylating compounds include but are not limited to, cisplatin, Paraplatin (carboplatin), eptaplatin, lobaplatin, nedaplatin, Eloxatin (oxaliplatin, Sanofi) or satrplatin and combinations thereof. Particularly preferred alkylating agents include Eloxatin (oxaliplatin).

Antimetabolites include but are not limited to, methotrexate, 6-mercaptopurine riboside, mercaptopurine, 5-fluorouracil (5-FU) alone or in combination with leucovorin, tegafur, LIFT, doxifluridine, carmofur, cytarabine, cytarabine ocfosfate, enocitabine, S-1, Alimta (premetrexed disodium, LY231514, MTA), Gemzar (gemcitabine, Eli Lilly), fludarabin, 5-azacitidine, capecitabine, cladribine, clofarabine, decitabine, eflornithine, ethynylcytidine, cytosine arabinoside, hydroxyurea, TS-1, melphalan, nelarabine, nolatrexed, ocfosfate, disodium premetrexed, pentostatin, pelitrexoi, raltitrexed, triapine, trimetrexate, vidarabine, vincristine, vinorelbine; or for example, one of the preferred anti-metaboiites disclosed in European Patent Application No. 239362 such as N-(5-[N-(3,4-dihydro-2-methyl-4-oxoquinazolin-6-ylmethyl)-N-methylamino]-2-thenoyl)-L-glutamic acid and combinations thereof.

Antibiotics include intercalating antibiotics but are not limited to: aclarubicin, actinomycin D, amrubicin, annamycin, adriamycin, bleomycin, daunorubicin, doxorubicin, elsamitrucin, epirubicin, galarubicin, idarubicin, mitomycin C, nemorubicin, neocarzinostatin, peplomycin, pirarubicin, rebeccamycin, stimalamer, streptozocin, valrubicin, zinostatin and combinations thereof.

Plant derived anti-tumor substances include for example those selected from mitotic inhibitors, for example vinblastine, docetaxel (Taxotere), paclitaxel and combinations thereof.

Cytotoxic topoisomerase inhibiting agents include one or more agents selected from the group consisting of aclarubicn, amonafide, belotecan, camptothecin, 10-hydroxycamptothecin, 9-aminocamptothecin, diflomotecan, irinotecan HCl (Camptosar), edotecarin, epirubicin (Eilence), etoposide, exatecan, gimatecan, lurtotecan, mitoxantrone, pirarubicin, pixantrone, rubitecan, sobuzoxane, SN-38, tafluposide, topotecan, and combinations thereof. Preferred cytotoxic topoisomerase inhibiting agents include one or more agents selected from the group consisting of camptothecin, 10-hydroxycamptothecin, 9-am inocamptothedn, irinotecan HCl (Camptosar), edotecarin, epirubicin (Eilence), etoposide, SN-38, topotecan, and combinations thereof.

Immunologicals include interferons and numerous other immune enhancing agents. Interferons include interferon alpha, interferon alpha-2a, interferon, alpha-2b, interferon beta, interferon gamma-1a, interferon gamma-1b (Actimmune), or interferon gamma-n1 and combinations thereof. Other agents include filgrastim, ientinan, sizofilan, TheraCys, ubenimex, WF-10, aldesleukin, alemtuzumab, BAM-002, dacarbazine, daclizumab, denileukin, gemtuzumab ozogamicin, ibritumomab, imiquimod, lenograstim, lentinan, melanoma vaccine (Corixa), molgramostim, OncoV AX-CL, sargramostim, tasonermin, tecleukin, thymalasin, tositumomab, Virulizin, 2-100, epratuzumab, mitumomab, oregovomab, pemtumomab (Y-muHMFGI), Provenge (Dendreon) and combinations thereof.

Biological response modifiers are agents that modify defense mechanisms of living organisms or biological responses, such as survival, growth, or differentiation of tissue cells to direct them to have anti-tumor activity. Such agents include krestin, lentinan, sizofiran, picibanil, ubenimex and combinations thereof.

Other anticancer agents include alitretinoin, ampligen, atrasentan bexarotene, bortezomib. Bosentan, calcitriol, exisuiind, finasteride.fotemustine, ibandronic acid, miltefosine, mitoxantrone, 1-asparaginase, procarbazine, dacarbazine, hydroxycarbamide, pegaspargase, pentostatin, tazarotne, Telcyta (TLK-286, Telik Inc.), Velcade (bortemazib, Millenium), tretinoin, and combinations thereof.

Other anti-angiogenic compounds include acitretin, fenretinide, thalidomide, zoledronic acid, angiostatin, aplidine, cilengtide, combretastatin A-4, endostatin, halofuginone, rebimastat, removab, Revlimid, squalamine, ukrain, Vitaxin and combinations thereof. Platinum-coordinated compounds include but are not limited to, cisplatin, carboplatin, nedaplatin, oxaliplatin, and combinations thereof.

Camptothecin derivatives include but are not limited to camptothecin, 10-hydroxycamptothecin, 9-aminocamptothecin, irinotecan, SN-38, edotecarin, topotecan and combinations thereof. Other antitumor agents include mitoxantrone, 1-asparaginase, procarbazine, dacarbazine, hydroxycarbamide, pentostatin, tretinoin and combinations thereof.

Anti-tumor agents capable of enhancing antitumor immune responses, such as CTLA4 (cytotoxic lymphocyte antigen 4) antibodies, and other agents capable of blocking CTLA4 may also be utilized, such as MDX-010 (Medarex) and CTLA4 compounds disclosed in U.S. Pat. No. 6,682,736; and anti-proliferative agents such as other farnesyl protein transferase inhibitors, for example the farnesyl protein transferase inhibitors.

Additionally, specific CTLA4 antibodies that can be used in the present invention include those described in U.S. Provisional Application 60/113,647 (filed Dec. 23, 1998), U.S. Pat. No. 6,682,736 both of which are herein incorporated by reference in their entirety.

Specific IGF1R antibodies that can be used in the present invention include those. described in International Patent Application No. WO 2002/053596, which is herein incorporated by reference in its entirety. Specific CD40 antibodies that can be used in the present invention include those described in International Patent Application No. WO 2003/040170 which is herein incorporated by reference in its entirety.

Gene therapy agents may also be employed as anti-tumor agents such as TNFerade (GeneVec), which express TNFalpha in response to radiotherapy.

In one embodiment of the present invention statins may be used in conjunction with a composition of the present invention. Statins (HMG-CoA reducatase inhibitors) may be selected from the group consisting of Atorvastatin (Lipitor, Pfizer Inc.), Pravastatin (Pravachol, Bristol-Myers Squibb), Lovastatin (Mevacor, Merck Inc.), Simvastatin (Zocor, Merck Inc.), Fluvastatin (Lescol, Novartis), Cerivastatin (Baycol, Bayer), Rosuvastatin (Crestor, AstraZeneca), Lovostatin and Niacin (Advicor, Kos Pharmaceuticals), derivatives and combinations thereof. In a preferred embodiment the statin is selected from the group consisting of Atovorstatin and Lovastatin, derivatives and combinations thereof. Other agents useful as anti-tumor agents include Caduet.

In one embodiment of the invention, the compositions of the present invention may be used in conjunction with photochemotherapy agents which are used to generate reactive oxygen species locally. Examples of photochemotherapy agents include palladium bacteriophephorbide (TOOKAD) used in photodynamic therapy; psoralen, 8-methoxypsoralen/methoxsalen (Oxsoralen-Ultra®, 8-MOP®, Oxsoralen®, Uvadex®), 4,5,8-trimethylpsoralen/trioxsalen (Trisoralen®), used in PUVA (Psoralen Ultra Violet A light); UVAR or UVAR® XTS™ Photopheresis System (Therakos, Inc., Exton, Pa.): Theraflex ECP® (Macopharma); CobeSpectra+Photo Immune System UVA PIT (Med Tech Solution); photosensitizers such as calcipotriene, tazarotene, chrysarobin and its synthetic derivative anthralin/1,8-dihydroxy-9-anthrone/dithranol (Drithocreme®); firefly (Photinus pyralis) luciferase used in (BioLuminescence Activated Destruction (BLADe)); erythrosin B (EB); erythrosine sodium; m-tetra(hydroxyphenyl)chlorin (m-THPC)/temoporfin (Foscan®, Biolitec AG); porphyrins such as d-aminolevulinic acid (d-ALA) (Levulan Kerastick®; DUSA Pharmaceuticals, Inc.), 5-ALA methylesther (MLA/M-ALA) (Metvix®; PhotoCure ASA), 5-ALA benzylesther (Benzvix®); 5-ALA hexylesther (Hexvix®), tin ethyl etiopurpurin (SnET2)/Sn etiopurpurin/rostaporfin (Photrex®, Purlytin®; Miravant MedicalTechnologies, boronated protoporphyrin (BOPP®), 2-(1-hexyloxyethyl)-2-divinyl pyropheophorbide-a (HPPH) (Photochlor®; Rosewell Park Cancer Institute), texaphyrins including europium texaphyrin (Eu-Tex), dysprosium texaphyrin (Dy-Tex), manganese texaphyrin (Mn-Tex), lutetium texaphyrin/PCI-0123 (Lu-Tex®, Lutex®, Lutrin®), motexafin lutetium (MLu)/lutetium(III) texaphyrin (Lu-Tex) (Antrin®, Lutrin®, Optrin®; Pharmacyclics Inc.), motexafin gadolinium (MGd)/PCI-0120 (Xcytrin®; source: Pharmacyclics Inc.) phthalocyanine-4 (Pc 4), taporfin sodium/NPe6/mono-L-aspartyl chlorin e6/taporfin sodium/LS11 (Talaporfin®; Light ScienceCorporation), benzoporphyrin derivative-monoacid ring A (BPD-MA)/verteporfin (Visudyne®, Novartis Pharmaceuticals), hematoporphyrin derivative (HpD) partially purified, porfimer sodium (Photofrin®; Axcan Pharma, Inc.), dihematoporphyrin ethers (DHE), photosan-3 (PS-3), photofrin-II, meso-tetrakis-phenylporphyrin (TPP) and tetraphenylporphinesulfonate (TPPS4)

### In Vitro Assays for Cell Killing

The cytotoxicity of the peptides on the prostate cancer derived cell lines DU145, LnCaP, and PC3, and the breast cancer derived cell line MDA-MB-231 was tested as previously described (Morgan et al. 2014 ibid), using the MTT (3-[4,5-dimethylthiazol-2-yl]-2,5-diphenyltetrazolium bromide) assay for metabolic activity. 7000-16000 cells, depending on the cell line, were seeded onto 96-well cell plate and treated with 5, 10, 20, 40 and 80 μM of each compound for 72h, DMSO alone (negative control) for 72 hr, or HTL001 (VVYKVVMKKAARRRRRRRRR; International patent application published as WO2017068353; positive control) for 2 h. After treatment, MTT at a final concentration of 0.5 mg/ml was added. Upon 4h incubation the formed formazan crystals were dissolved in DMSO and the optical density (OD) measured at 540 nm with a spectrofluorimeter. Percentage of cell survival was calculated as a ratio of the mean OD value of treated vs. untreated cells. Experiments were repeated 3 times and statistical analysis performed using Student's t-test. All cell lines were cultured in RPMI media with added 10% FBS, 1% sodium pyruvate and 1% L-Glutamine. They were all cultured in an incubator at 37° C., 5% CO₂. The assay was repeated 3 times and the results are given as the mean IC50 for cell killing±standard deviation.

The following Examples were tested:

| | Designated | Molecular |
|---|---|---|
| Structure | number | weight |
| 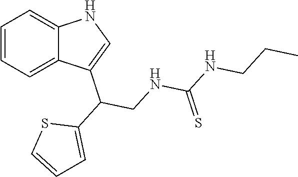 [SMILES: CCCNC(=S)NCC(c1cccs1)c1c[nH]c2ccccc12] | Example 8 | 343.521 |
| 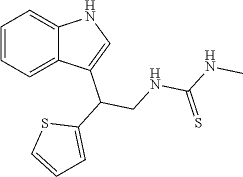 [SMILES: CNC(=S)NCC(c1cccs1)c1c[nH]c2ccccc12] | Example 6 | 315.467 |
| 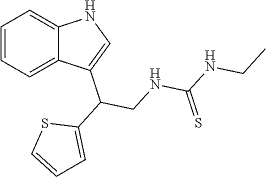 [SMILES: CCNC(=S)NCC(c1cccs1)c1c[nH]c2ccccc12] | Example 7 | 333.51 |
| 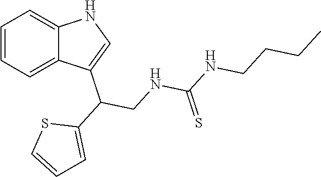 [SMILES: CCCCNC(=S)NCC(c1cccs1)c1c[nH]c2ccccc12] | Example 9 | 357.548 |
| 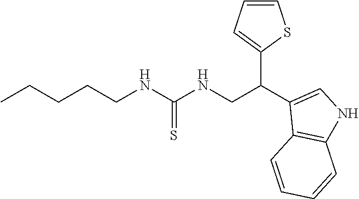 [SMILES: CCCCCNC(=S)NCC(c1cccs1)c1c[nH]c2ccccc12] | Example 3 | 371 |
| 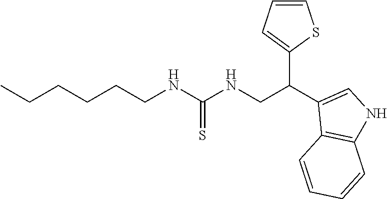 [SMILES: CCCCCCNC(=S)NCC(c1cccs1)c1c[nH]c2ccccc12] | Example 2 | 385 |
| 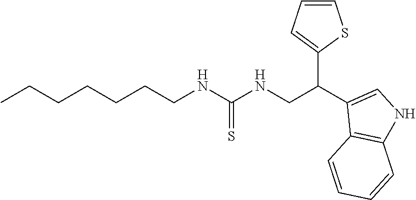 [SMILES: CCCCCCCNC(=S)NCC(c1cccs1)c1c[nH]c2ccccc12] | Example 1 | 399 |
| 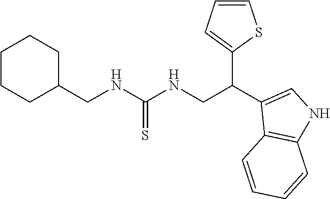 [SMILES: S=C(NCC1CCCCC1)NCC(c1cccs1)c1c[nH]c2ccccc12] | Example 4 | 397 |
| 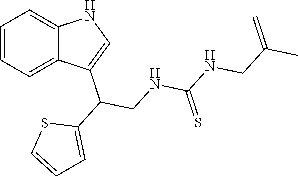 [SMILES: C=C(C)CNC(=S)NCC(c1cccs1)c1c[nH]c2ccccc12] | Example 5 | 355.532 |

In addition, the following reference examples were tested. The reference examples are urea analogues of the 2-(1H-indol-3-yl)-2-(2-thienyl)ethyl]thiourea compounds of formula (I)

| | Desig- | Molecu- |
|---|---|---|
| | nated | lar |
| Structure | number | weight |
| 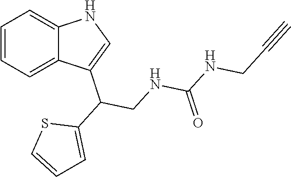 [SMILES: C#CCNC(=O)NCC(c1cccs1)c1c[nH]c2ccccc12] | A | 323.421 |
| 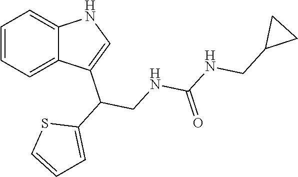 [SMILES: O=C(NCC1CC1)NCC(c1cccs1)c1c[nH]c2ccccc12] | B | 339.464 |
| 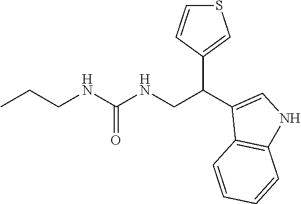 [SMILES: CCCNC(=O)NCC(c1ccsc1)c1c[nH]c2ccccc12] | C | 327 |
| 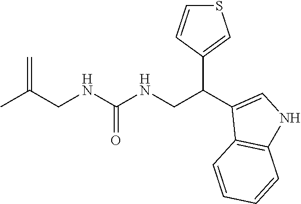 [SMILES: C=C(C)CNC(=O)NCC(c1ccsc1)c1c[nH]c2ccccc12] | D | 339 |

The molecules were tested for their ability to induce cell death using the breast cancer cell line MDA-MB-231, with the HOX/PBX interfering peptide HTL001 (WYKWMKKAARRRRRRRRR; International patent application published as WO2017068353) as a positive control and DMSO as a negative control. Percentage of cell survival was evaluated with the MTT assay after treatment with different concentrations (2, 10, 20, 40 and 80 μM) of each compound for 72h. The IC50 was evaluated as the concentration of the compound that reduces % of cell survival by half. IC50 data for Examples 1, 2, 3, 4, 5, 6, 7, 8 and 9 are shown below.

| | Exp1 | Exp2 | Exp3 | Exp4 | Exp5 | Average | SEM |
|---|---|---|---|---|---|---|---|
| Example 8 | 42.72 | 48.65 | 56.10 | 59.91 | | 51.85 | 4.43 |
| Example 6 | 67.97 | 47.95 | 68.94 | | | 61.62 | 6.84 |
| Example 7 | 51.81 | 65.82 | 64.97 | | | 60.87 | 4.53 |
| Example 9 | 40.94 | 37.308097 | 29.816573 | 40.99294 | 57.182569 | 41.25 | 7.07 |
| Example 3 | 21.32 | 49.201 | | | | 35.26 | |
| Example 2 | 36.73 | 38.974 | | | | 37.85 | |
| Example 1 | 21.83 | 44.278 | | | | 33.05 | |
| Example 4 | 65.81 | 40.259 | | | | 53.03 | |
| Example 5 | 47.93 | 44.95 | 43.70 | | | 45.53 | 1.26 |

With regards reference examples A, B, C. D and E, it was not possible at a concentration of 80 μM to observe induced cell death and measure IC50 values. Thus, IC50 values for the urea analogue reference examples A, B, C and D are greater than 80 μM

### Cell Based Assay for HOX/PBX Binding

A suitable assay for determining the antagonism activity of a compound in relation to the interaction between HOX and PBX is described hereinbelow.

In order to directly assess the ability of each compound to disrupt HOX/PBX binding a cell-based assay system was developed that could allow the formation of HOX/PBX/DNA dimers. Cultured MDA-MB-231 cells were treated with 10 μM of each compound for 4 hours and then used to generate a cell lysate using a standard preparation method as described below. HOXB4/PBX2 dimers were then measured using an ELISA-based system as detailed in the table below. The values from this assay are expressed as % inhibition of dimer formation compared to a negative control (DMSO).

### Lysate Preparation:

1. Collect approximately 5.0×107 cells by low-speed centrifugation at RT for 5 min. Carefully remove culture medium.
2. Wash the cellular debris (pellet) with PBS at RT, and collect by low-speed centrifugation. Carefully remove supernatant (total protein).
3. Add 1.0 ml of pre-cold RIPA buffer (or other appropriate buffer) with freshly added (Protease Inhibitors) and/or (Phosphatase Inhibitors). Gently resuspend cells in RIPA buffer with a pipet and incubate on ice for 30 min.
4. Further disrupt and homogenize cells by passing through a 21-gauge needle, dounce homogenization or sonication, taking care not to raise the temperature of the lysate. (Optional: Add 10 μl of 10 mg/ml PMSF stock) Incubate 30 min on ice.
5. Transfer to microcentrifuge tube(s) and centrifuge at 10,000×g for 10 min at 4° C. The supernatant fluid is the total cell lysate. Transfer the supernatant to a new microfuge tube and discard the pellet.

### Assay Method:

All volumes 0.1 ml unless specified; RT. Plates washed by submersion.

| | | Time | |
|---|---|---|---|
| Step | Process | (mins) | Resource |
| 1 | Coat plates | 60 | 0.2 mg/ml streptavidin (Sigma 85878 |
| | | | 1 mg, dissolve in 5 ml PBST) |
| 2 | Wash | x4 | PBST |
| 3 | Anti-mouse Ab | 30 | Donkey anti-mouse-biotin, ab7060, use |
| | biotin | | at 1:1000 |
| 4 | Wash | x4 | PBST |
| 5 | Anti-PBX2 | 30 | mAb to PBX2, ab55498, use at 1:500 |
| 6 | Wash | x4 | PBST |
| 7 | Block | 60 | T20 |
| 8 | Wash | x4 | PBST |
| 7 | Cell lysate | 30 | Diluted to 100 μg/ml in PBST |
| 8 | Wash | x4 | PBST |
| 9 | Anti-HOXB4 | 30 | Rabbit anti-HOXB4, ab56049, |
| | | | use at 1:10,000 |
| 10 | Wash | x4 | PBST |
| 11 | Anti-rabbit AP | 30 | Goat anti-rabbit AP, ab6722, use |
| | | | at 1:3000 |
| 12 | Wash | x4 | PBST |
| 13 | Wash | x1 | TBS |
| 14 | Colour | 30 | pNpp solution |
| | development | | |

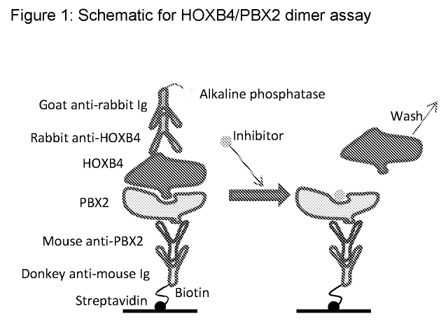

A schematic diagram for HOXB4/PBX2 dimer assay is shown in FIG. 1.

Example 5 showed a 39.2 (SEM 5.7) % inhibition of HOXB4/PBX2 dimer formation.

cFOS Expression in MDA-MB-231 Cells

cFos and DUSP1 have previously been shown to be biomarkers for tumor cell response to HOX/PBX inhibition (Morgan, R., Pirard, P. M., Shears, L., Sohal, J., Pettengell, R. & Pandha, H. S. (2007) Antagonism of HOX/PBX dimer formation blocks the in vivo proliferation of melanoma. Cancer Res, 67, 5806-5813).

MDA-MB-231 cells were seeded in 25 cm² plates and treated for 2h with 33 μM of each compound at 80% confluence. RNA was isolated using RNeasy Mini Kit-QIAGEN according to manufacturer's instructions and quantified with NanoDrop ND-1000. cDNA was generated from 1 μg of total RNA using High Capacity cDNA Reverse Transcription Kit in a 25 μl final reaction volume according to the manufacturer protocol. The expression of cFOS was quantified by qRT-PCR. Real-time PCR reactions were performed using 1:10 dilution (5 μl/well) of each cDNA added to TaqMan Universal PCR Master Mix and TaqMan Gene Expression Assay Hs00170630_m1 FOS. Amplification of glyceraldehyde 3-phosphate dehydrogenase (GAPDH) probe was performed as endogenous control. The comparative Ct method (ΔΔCt algorithm) was used for analysis. Independent experiments were performed in triplicates and repeated three times. Statistical analysis was performed with Student's t-test.

Example 1 showed a 32.5 fold induction of cFos

Example 5 showed a 37.4 (SEM 3.4) fold induction of cFos

Examples 1 and 5 caused a significant increase in cFos expression compared to untreated MDA-MB-231 cells

These results indicate that cFos can act as a biomarker of tumour response to a compound of the invention, and as such its elevated expression is a potential surrogate clinical trial endpoint.

### In Vivo Studies

MDA-MB-231 cells were injected subcutaneously into female or male Balb-c nude mice aged 6 to 12 weeks (Harlan, UK). When the tumour size reached 100 mm³ mice were injected intratumouraly with Example 2 at the intervals shown on the graph. Mice were sacrificed when the tumour size reached 1000 mm³ or at the end of the experiment (14 days).

Treatment of MDA-MB-231 tumour with Example 2 (ICT9119):
Group 1—Untreated controls
Group 2—10% DMSO/Oil solvent control, IP, day 0, 3, 6, 9, 10 & 13
Group 3—Example 2, 20 mg/Kg, IP, day 0, 3, 6, 9, 10 & 13

### Results

| Group | Median time | Growth delay | | Maximum % |
|---|---|---|---|---|
| number | RTV 2 (days) | (days) | Significance | weight loss |
| 1 | 3.8 | — | — | 0 |
| 2 | 4.7 | 0.9 | p > 0.05 ns | 0 |
| 3 | 8.9 | 4.2 | p = 0.01 | 0 |

No statistically significant difference was observed between solvent and untreated group.

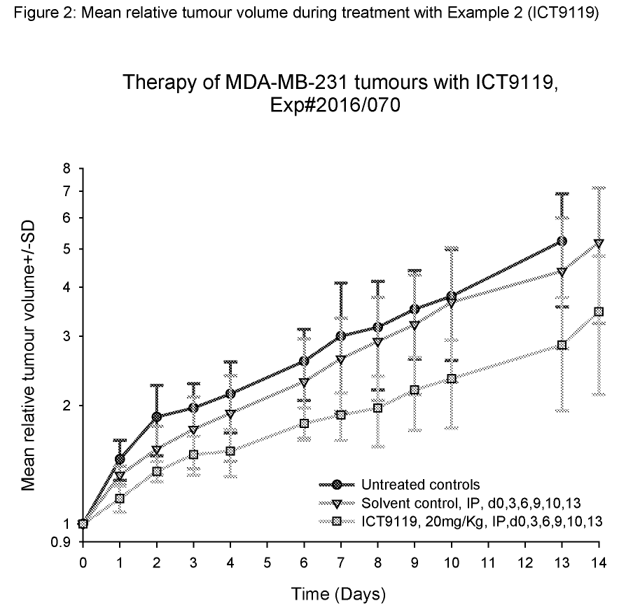

Mean relative tumour volume during treatment with Example 2 (ICT9119) is shown in FIG. 2.

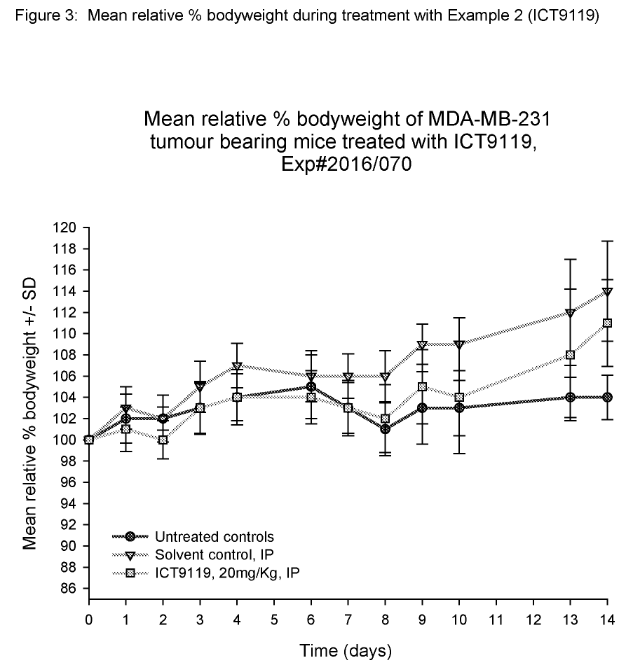

Mean relative % bodyweight during treatment with Example 2 (ICT9119) is shown in FIG. 3.

### Solubility Studies

Example 2 was solubilised in PEG400 and propylene glycol (PG). Higher concentrations were achieved with PG (up to 5 mg/mL). The compound remained in solution when diluted with water with the addition of polysorbate 80 (Tween 80) as a surfactant at a final concentration of 5%.

A stock formulation in PEG400 for use in in vitro assays was prepared as follows:

1 mg of Example 2 was dissolved in 1 mL of PEG400. 1 mL of 90% water 10% Tween 80 (v/v) was added with vortexing. Concentration of the stock formulation was 0.5 mg/mL in 50% PEG400, 45% water, 5% Tween 80. The stock formulation was diluted as required in medium.

### MDA-MB-231 Cell Survival

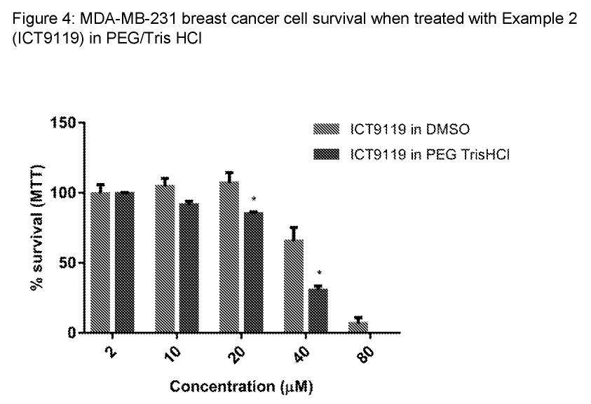

As shown in FIG. 4, Example 2 (ICT9119) was more effective at killing breast cancer-derived MDA-MB-231 cells when dissolved in PEG400.

MDA-MB-231 cells were treated with different concentrations of Example 2 dissolved in either DMSO or PEG/Tris HCl and MTT assay performed. Data are means±SEM of four independent experiments. MDA-MB-231 cell survival was significantly decreased when treated with Example 2 (ICT9119) in PEG/Tris HCl in comparison with Example 2 (ICT9119) in DMSO (*p<0.05; Ttest).

cFOS Expression in MDA-MB-231 Cells

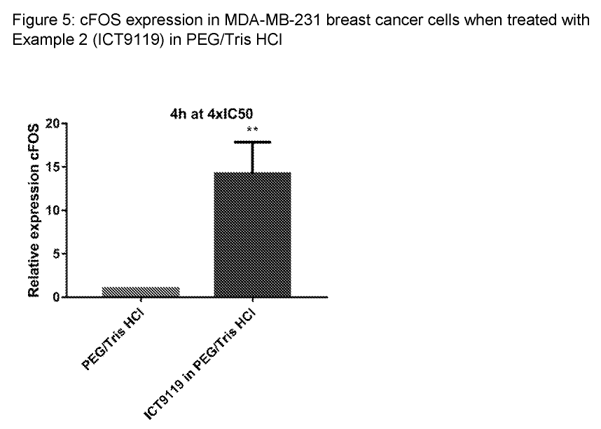

As shown in FIG. 5, Example 2 (ICT9119) in PEG/Tris HCl significantly increased cFOS expression in MDA-MB-231 breast cancer cells after 4h treatment.

MDA MB 231 cells were treated for 4h with 4×IC50 Example 2 in PEG/Tris HCl. RNA was isolated and RT-PCR performed to detect expression of cFOS. Data are means±SEM of four independent experiments. Significance to PEG/Tris HCl: **p<0.01 (Ttest).

cFOS and DUSP1 Expression in PC3 Prostate Cancer Cells

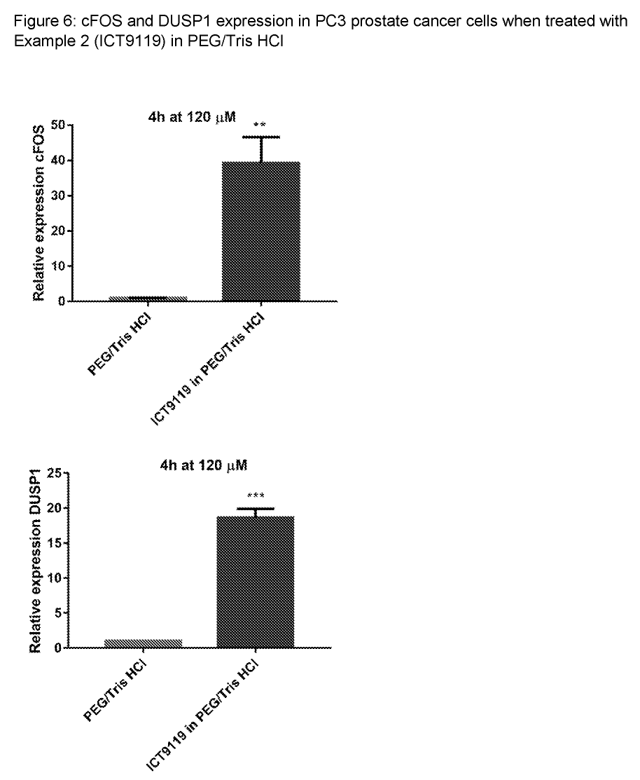

As shown in FIG. 6, Example 2 (ICT9119) in PEG/Tris HCl significantly increased cFOS and DUSP1 expression in PC3 prostate cancer cells.

PC3 cells were treated for 4h with 120 μM Example 2 in PEG/Tris HCl. RNA was isolated and RT-PCR performed to detect expression of cFOS and DUSP1. Data are means±SEM of three independent experiments. Significance to PEG/Tris HCl: **p<0.01, ***p<0.001 (Ttest).

cFOS and DUSP1 Expression in DU145 Prostate Cancer Cells

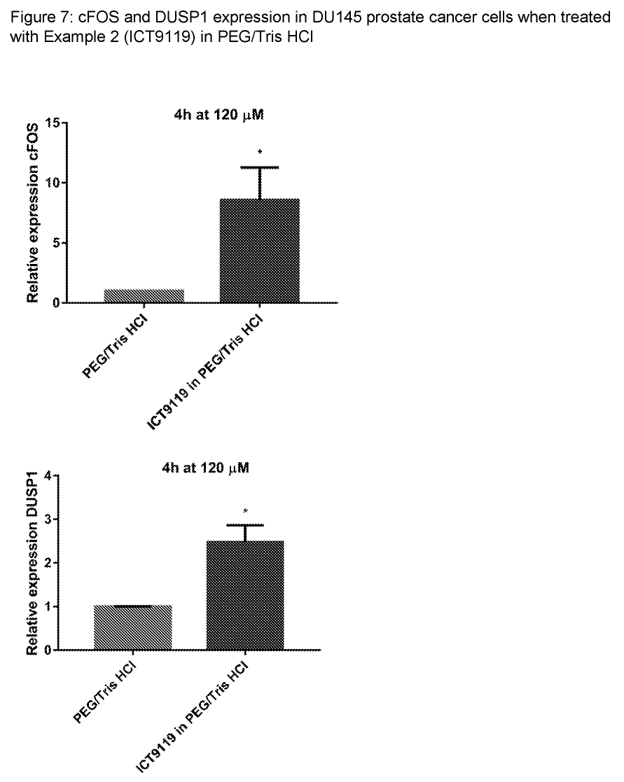

As shown in FIG. 7, Example 2 (ICT9119) in PEG/Tris HCl significantly increased cFOS and DUSP1 expression in DU145 prostate cancer cells.

DU145 cells were treated for 4h with 120 μM Example 2 in PEG/Tris HCl. RNA was isolated and RT-PCR performed to detect expression of cFOS and DUSP1. Data are means±SEM of three independent experiments. *p<0.05 (Ttest).

The following Examples illustrate the invention:

### EXAMPLES

Referring to the examples that follow, compounds of the preferred embodiments are synthesized using the methods described herein, or other methods, which are known in the art.

It should be understood that the organic compounds according to the preferred embodiments may exhibit the phenomenon of tautomerism. As the chemical structures within this specification can only represent one of the possible tautomeric forms, it should be understood that the preferred embodiments encompasses any tautomeric form of the drawn structure.

It is understood that the invention is not limited to the embodiments set forth herein for illustration, but embraces all such forms thereof as come within the scope of the above disclosure.

### General Conditions:

The following examples are intended to illustrate the invention and are not to be construed as being limitations thereon. Temperatures are given in degrees centigrade. If not mentioned otherwise, all evaporations are performed under reduced pressure. The structure of final products, intermediates and starting materials is confirmed by standard analytical methods, e.g., microanalysis and spectroscopic characteristics, e.g., MS, IR, NMR. Abbreviations used are those conventional in the art. If not defined, the terms have their generally accepted meanings.

### Abbreviations

bs broad singlet
d doublet
d(n) day(number of day)
DCM dichloromethane
DMF N,N-dimethylformamide
DMSO dimethylsulfoxide
DTE ethane ditriflate (2-(trifluoromethylsulfonyloxy)ethyl trifluoromethanesulfonate)
h hour(s)
i.p. intraperitoneal
i.v intraveneous
MeOH methanol
MS-ES+ mass spectrometry-electrospray
m multiplet
min minutes
ml milliliter(s)
m/z mass to charge ratio
NMR nuclear magnetic resonance
ppm parts per million
s singlet
SVVV square wave voltammagram
t triplet
TEA triethylamine
TFA trifluoroacetic acid
THF tetrahydrofuran

Referring to the examples that follow, compounds of the preferred embodiments were synthesized using the methods described herein, or other methods, which are known in the art.

The various starting materials, intermediates, and compounds of the preferred embodiments may be isolated and purified, where appropriate, using conventional techniques such as precipitation, filtration, crystallization, evaporation, distillation, and chromatography. Unless otherwise stated, all starting materials are obtained from commercial suppliers and used without further purification. Salts may be prepared from compounds by known salt-forming procedures.

It should be understood that the organic compounds according to the preferred embodiments may exhibit the phenomenon of tautomerism. As the chemical structures within this specification can only represent one of the possible tautomeric forms, it should be understood that the preferred embodiments encompasses any tautomeric form of the drawn structure.

Example compounds of the present invention include:

### Example 1

### 1-heptyl-3-[2-(1H-indol-3-yl)-2-(2-thienyl)ethyl]thiourea

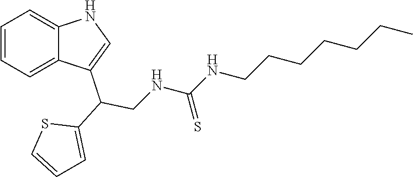 [SMILES: CCCCCCCNC(=S)NCC(c1cccs1)c1c[nH]c2ccccc12]

To a stirred solution of 2-(1H-indol-3-yl)-2-(thiophen-2-yl)ethan-1-amine (30 mg, 0.124 mmol) in THF (3 mL) was added DIPEA (22 μL, 16 mg, 0.124 mmol) and heptyl isothiocyanate (21 μL, 20 mg, 0.124 mmol) and the reaction mixture heated to reflux for 22.5 h. The mixture was cooled to room temperature, concentrated in vacuo and purified by flash column chromatography (EtOAc:PE, 1:2) to yield the title compound (37 mg, 75%) as a light brown oil which darkened on standing; Rf 0.53 (EtOAc:PE, 1:1); δ_{H} (400 MHz, CDCl₃) 8.25 (1H, brs), 7.58 (1H, d, J 8.1), 7.38 (1H, d, J 8.1), 7.19 (1H, dt, J 1.0, 7.1), 7.18 (1H, dd, J 1.5, 5.0), 7.14 (1H, d, J 2.0), 7.08 (1H, dt, J 1.0, 7.1), 6.98 (1H, td, J 1.0, 5.0), 6.50 (1H, dd, J 3.5, 5.1), 5.78 (1H, br), 5.71 (1H, br), 4.91 (1H, t, J 7.1), 4.27 (1H, br), 4.18 (1H, br), 3.05 (2H, br), 1.66-1.72 (2H, m), 1.18-1.42 (8H, m), 0.88 (3H, t, J 7.1); δ_{C} (100 MHz, CDCl₃) 183.5 (C), 147.6 (C), 136.5 (C), 126.9 (CH), 126.4 (C), 125.0 (CH), 124.2 (CH), 122.5 (CH), 122.2 (CH), 119.9 (CH), 117.4 (C), 111.4 (CH), 45.1 (CH₂), 37.8 (CH), 31.6 (CH₂), 30.0 (CH₂), 28.8 (CH₂), 28.6 (CH₂), 26.8 (CH₂), 22.6 (CH₂), 14.1 (CH₃); m/z (AP+) 400 (M+H⁺, 3%), 117 (100), 88 (23); HRMS 398.1721 [M−2H+H]⁺; C₂₂H₂₈N₃S₂ req. 398.1719.

### Example 2

### 1-hexyl-3-[2-(1H-indol-3-yl)-2-(2-thienyl)ethyl]thiourea

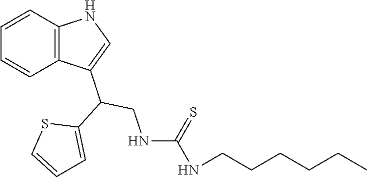 [SMILES: CCCCCCNC(=S)NCC(c1cccs1)c1c[nH]c2ccccc12]

To a stirred solution of 2-(1H-indol-3-yl)-2-(thiophen-2-yl)ethan-1-amine (31 mg, 0.128 mmol) in THF (3 mL) was added DIPEA (22 μL, 17 mg, 0.128 mmol) and hexyl isothiocyanate (20 μL, 18 mg, 0.128 mmol) and the reaction mixture heated to reflux for 21.5 h. The mixture was cooled to room temperature, concentrated in vacuo and purified by flash column chromatography (EtOAc:PE, 1:2) to yield the title compound (34 mg, 69%) as a brown oil; Rf 0.51 (EtOAc:PE, 1:1); δ_{H} (400 MHz, CDCl₃) 0.26 (1H, brs), 7.58 (1H, d, J 8.1), 7.37 (1H, d, J 8.1), 7.19 (1H, dt, J 1.0, 7.1), 7.18 (1H, dd, J 1.5, 5.0), 7.13 (1H, brd, J 2.0), 7.08 (1H, dt, J 1.0, 7.1), 6.98 (1H, td, J 1.0, 3.5), 6.95 (1H, dd, J 3.5, 5.0), 5.80 (1H, br), 5.72 (1H, br), 4.90 (1H, t, J 7.3), 7.26 (1H, br), 4.17 (1H, br), 3.05 (2H, br), 1.19-1.38 (8H, m), 0.86 (3H, t, J 7.1); δ_{C} (100 MHz, CDCl₃) 159.9 (C), 145.8 (C), 136.5 (C), 126.9 (CH), 126.4 (C), 125.0 (CH), 124.2 (CH), 122.5 (CH), 122.2 (CH), 119.8 (CH), 119.4 (CH), 116.2 (C), 111.4 (CH), 50.0 (CH₂), 37.8 (CH), 31.3 (CH₂), 28.6 (CH₂), 26.5 (CH₂), 22.5 (2CH₂), 14.0 (CH₃); m/z (AP+) 386 (M+H+, 100); HRMS 386.1711; C₂₁H₂₈N₃S₂ req. 386.1719.

### Example 3

### 1-pentyl-3-[2-(1H-indol-3-yl)-2-(2-thienyl)ethyl]thiourea

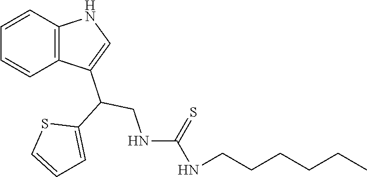 [SMILES: CCCCCCNC(=S)NCC(c1cccs1)c1c[nH]c2ccccc12]

To a stirred solution of 2-(1H-indol-3-yl)-2-(thiophen-2-yl)ethan-1-amine (34 mg, 0.140 mmol) in THF (3 mL) was added DIPEA (24 μL, 18 mg, 0.140 mmol) and pentyl isothiocyanate (18 μL, 16 mg, 0.140 mmol) and the reaction mixture heated to reflux for 19 h. The mixture was cooled to room temperature, concentrated in vacuo and purified by flash column chromatography (EtOAc:PE, 1:1) to yield the title compound (72 mg) as a brown oil; Rf 0.45 (EtOAc:PE, 1:1); δ_{H} (400 MHz, CDCl₃) 8.24 (1H, brs), 7.58 (1H, d, J 8.1), 7.38 (1H, dd, J 1.0, 8.1), 7.18-7.22 (2H, m), 7.15 (1H, brs), 7.09 (1H, dt, J 1.0, 7.1), 6.99 (1H, m), 6.95 (1H, dd, J 3.5, 5.0), 5.80 (1H, vvvbr), 4.90 (1H, t, J 7.1), 4.23 (1H, brm), 4.09-4.18 (1H, brm), 3.04 (2H, br), 1.37-1.43 (3H, m), 1.16-1.26 (5H, m), 0.79 (3H, t, J 7.1); δ_{C} (100 MHz, CDCl₃) 180.3 (C), 145.6 (C), 136.5 (C), 126.9 (CH), 126.4 (C), 124.9 (CH), 124.2 (CH), 122.6 (CH), 122.2 (CH), 119.9 (CH), 119.4 (CH), 114.9 (C), 111.4 (CH), 46.9 (CH₂), 37.8 (CH), 34.6 (CH₂), 28.9 (CH₂), 28.3 (CH₂), 22.2 (CH₂), 13.9 (CH₃); m/z (AP+) 372 (M+H+, 9%), 117 (100), 88 (47); HRMS 370.1404 [M−2H+H]+; C₂₀H₂₄N₃S₂req 370.1406.

### Example 4

### 1-cyclohexylmethyl-3-[2-(1H-indol-3-yl)-2-(2-thienyl)ethyl]thiourea

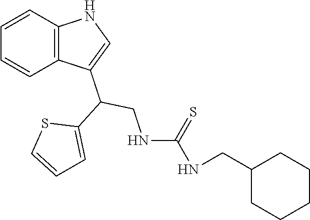 [SMILES: S=C(NCC1CCCCC1)NCC(c1cccs1)c1c[nH]c2ccccc12]

To a stirred solution of 2-(1H-indol-3-yl)-2-(thiophen-2-yl)ethan-1-amine (37 mg, 0.153 mmol) in THF (4 mL) was added DIPEA (27 μL, 20 mg, 0.153 mmol) and cyclohexylmethyl isothiocyanate (24 μL, 24 mg, 0.153 mmol) and the reaction mixture heated to reflux for 23.5 h. The mixture was cooled to room temperature, concentrated in vacuo and purified by flash column chromatography (EtOAc:PE, 1:4→1:2) to yield the title compound (49 mg, 81%) as a colourless oil/glass which darkened on standing; Rf 0.67 (EtOAc:PE, 1:1); δ_{H} (400 MHz, CDCl₃) 8.29 (1H, brs), 7.58 (1H, d, J 8.1), 7.37 (1H, d, J 8.1), 7.19 (1H, dt, J 1.0, 8.1), 7.17 (1H, dd, J 1.5, 5.1), 7.11 (1H, br), 7.08 (1H, dt, J 1.0, 7.1), 6.97 (1H, dd, J 1.0, 3.5), 6.95 (1H, dd, J 3.5, 5.0), 5.776 (2H, vbr), 4.89 (1H, t, J 7.1), 4.25 (1H, br), 4.17 (1H, br), 2.88 (2H, br), 1.55-1.66 (5H, brm), 1.24-1.29 (2H, m), 1.05-1.18 (2H, m), 0.76 (2H, brq, J 11.6); δ_{C} (100 MHz, CDCl₃) 145.8 (C), 136.5 (C), 126.8 (CH), 126.4 (C), 124.9 (CH), 124.2 (CH), 122.5 (CH), 122.2 (CH), 119.9 (CH), 119.4 (CH), 116.2 (C), 111.4 (CH), 50.2 (CH₂), 37.8 (CH), 37.3 (CH), 30.8 (CH₂), 26.2 (CH₂), 25.6 (CH₂); m/z (AP+) 398 (M+H+, 69%), 285 (98), 243 (100); HRMS 396.1562 [M−2H+H]⁺; C₂₂H₂₆N₃S₂ req. 396.1563.

1. A compound of Formula I: 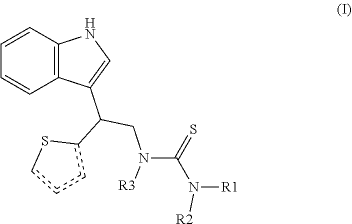
or a pharmaceutically salt or solvate thereof, wherein
R¹ is H, C₁-C₈ alkyl; C₃-C₁₀ cycloalkyl; C₂-C₈ alkenyl; C₅-C₁₀-cycloalkenyl; C₂-C₈ alkynyl; C₆-C₁₅ aryl; (C₁-C₄ alkyl)-(C₃-C₁₀ cycloalkyl); (C₁-C₄ alkyl)-(C₂-C₈ alkenyl); (C₁-C₄ alkyl)-(C₅-C₁₀ cycloalkenyl); (C₁-C₄ alkyl)-(C₂-C₈ alkynyl); or (C₁-C₄ alkyl)-(C₆-Cis aryl); and
R² and R³ are independently selected from H and C₁-C₃alkyl;
and [symbol] denotes a single or double bond, with the proviso that a maximum of two double bonds are present, which cannot be adjacent to each other.

2. The compound according to claim 1 or a pharmaceutically salt or solvate thereof wherein R¹ is C₁-C₈ alkyl; C₃-C₁₀ cycloalkyl; C₂-C₈ alkenyl; C₅-C₁₀-cycloalkenyl; C₂-C₈ alkynyl; (C₁-C₄ alkyl)-(C₃-C₁₀ cycloalkyl); (C₁-C₄ alkyl)-(C₂-C₈ alkenyl); (C₁-C₄ alkyl)-(C₅-C₁₀ cycloalkenyl); or (C₁-C₄ alkyl)-(C₂-C₈ alkynyl).

3. The compound according to claim 1 or a pharmaceutically salt or solvate thereof wherein R¹ is C₁-C₈ alkyl; (C₁-C₄ alkyl)-(C₃-C₈ cycloalkyl); (C₁-C₄ alkyl)-(C₂-C₈ alkenyl); (C₁-C₄ alkyl)-(C₅-C₁₀ cycloalkenyl); or (C₁-C₄ alkyl)-(C₂-C₈ alkynyl).

4. The compound according to claim 1 or a pharmaceutically salt or solvate thereof, wherein R¹ is C₁-C₈ alkyl; (C₁-C₄ alkyl)-(C₃-C₈ cycloalkyl); or (C₁-C₄ alkyl)-(C₂-C₈ alkenyl).

5. The compound according to claim 1 or a pharmaceutically salt or solvate thereof, wherein R¹ is C₁-C₈ alkyl.

6. The compound according to claim 1 or a pharmaceutically salt or solvate thereof, wherein R¹ is C₅-C₇ alkyl.

7. The compound according to claim 1 or a pharmaceutically salt or solvate thereof, wherein R¹ is (C₁-C₄ alkyl)-(C₂-C₈ alkenyl)

8. The compound according to claim 1 or a pharmaceutically salt or solvate thereof, wherein R¹ is (C₁-C₄ alkyl)-(C₃-C₈ cycloalkyl)

9. The compound according to claim 1 or a pharmaceutically salt or solvate thereof, wherein R² is H.

10. The compound according to claim 1 or a pharmaceutically salt or solvate thereof, wherein R³ is H.

11. The compound of claim 1, wherein the compound is of Formula I′: 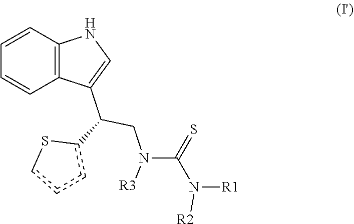
or a pharmaceutically salt or solvate thereof.

12. The compound of claim 1, wherein the compound is of Formula Ia: 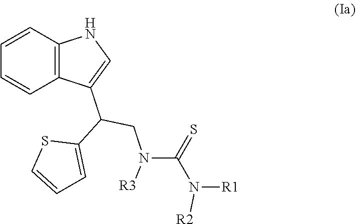
or a pharmaceutically salt or solvate thereof.

13. The compound of claim 1, wherein the compound is of Formula Ia′: 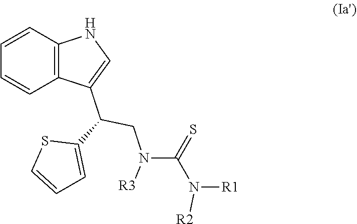
or a pharmaceutically salt or solvate thereof.

14. The compound of claim 1, wherein the compound is of Formula Ib: 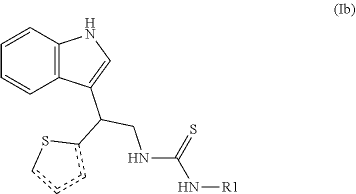
or a pharmaceutically salt or solvate thereof.

15. The compound of claim 1, wherein the compound is of Formula Ib′: 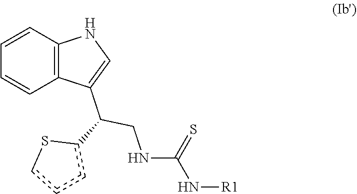
or a pharmaceutically salt or solvate thereof.

16. The compound of claim 1, wherein the compound is of Formula Ic: 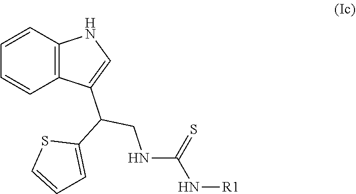
or a pharmaceutically salt or solvate thereof.

17. The compound of claim 1, wherein the compound is of Formula Ic′: 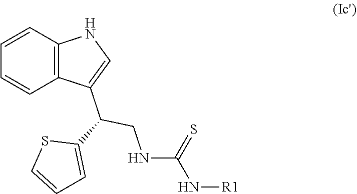
or a pharmaceutically salt or solvate thereof.

18. The compound according to claim 1 wherein said compound is selected from:
1-heptyl-3-[(2-(1H-indol-3-yl)-2-(2-thienyl)ethyl]thiourea;
1-hexyl-3-[(2-(1H-indol-3-yl)-2-(2-thienyl)ethyl]thiourea;
1-pentyl-3-[(2-(1H-indol-3-yl)-2-(2-thienyl)ethyl]thiourea;
1-butyl-3-[(2-(1H-indol-3-yl)-2-(2-thienyl)ethyl]thiourea;
1-propyl-3-[2-(1H-indol-3-yl)-2-(2-thienyl)ethyl]thiourea;
1-ethyl-3-[(2-(1H-indol-3-yl)-2-(2-thienyl)ethyl]thiourea;
1-methyl-3-[2-(1H-indol-3-yl)-2-(2-thienyl)ethyl]thiourea;
1-cyclohexylmethyl-3-[2-(1H-indol-3-yl)-2-(2-thienyl)ethyl]thiourea; and
1-[2-(1H-indol-3-yl)-2-(2-thienyl)ethyl]-3-(2-methylallyl)thiourea;
or a pharmaceutically acceptable salt or solvate thereof.

19. A pharmaceutical composition comprising the compound according to claim 1 or a pharmaceutically salt or solvate thereof, and one or more pharmaceutically acceptable excipients, diluents and/or carriers.

20. The pharmaceutical composition according to claim 19 in combination with one or more other therapeutic agents.

21. The pharmaceutical composition according to claim 20 wherein the one or more other therapeutic agents comprises one or more cytotoxic agents, one or more chemotherapeutic agents, or a combination thereof.

22. (canceled)

23. A method for treating or preventing a condition or disorder in which aberrant cell division occurs, the method comprising administering to a subject in need thereof a therapeutically effective amount of the compound of claim 1 or a pharmaceutically salt or solvate thereof.

24. The method according to claim 23 wherein said condition or disorder is a cancer.

25. The method according to claim 23, wherein the subject is also administered a cytotoxic or chemotherapeutic agent.

26. The method according to claim 23 wherein said cells express one or more Hox genes.

27. The method according to claim 23, wherein PBX does not act as an oncogene in said cells.

28. A method for reducing the side effects of a cytotoxic or chemotherapeutic agent, the method comprising administering to a subject in need thereof a therapeutically effective amount of the compound of claim 1 or a pharmaceutically salt or solvate thereof.

29. A method for maintaining or expanding a stem cell population in vivo, the method comprising administering to a subject in need thereof a therapeutically effective amount of the compound of claim 1 or a pharmaceutically salt or solvate thereof.

30-32. (canceled)

33. A method of maintaining or expanding stem cells ex vivo, the method comprising contacting said stem cells with a compound as defined in claim 1 or a pharmaceutically salt or solvate thereof.
